(19) United States
(12) Patent Application Publication
(10) Pub. No.: US 2024/0198039 A1
(43) Pub. Date: Jun. 20, 2024

(54) CATHETERS WITH LASER CUT HYPOTUBE SCAFFOLD FOR FLOW UNDER SIGNIFICANT PRESSURE DIFFERENTIALS

(71) Applicant: MIVI Neuroscience, Inc., Eden Prairie, MN (US)

(72) Inventors: John Wainwright, Foothill Ranch, CA (US); John VanScoy, Plymouth, MN (US); Jack B. Sattell, Cambridge, MA (US)

(21) Appl. No.: 18/539,695

(22) Filed: Dec. 14, 2023

Related U.S. Application Data
(60) Provisional application No. 63/432,874, filed on Dec. 15, 2022.

(51) Int. Cl. A61M 25/00; A61M 25/01

(52) U.S. Cl. CPC A61M 25/0013; A61M 25/0014; A61M 25/0045; A61M 25/0108

(57) ABSTRACT

Medical catheters for transfer of fluids comprise a laser cut hypotube and an embedded polymer flowed into the cuts of the laser-cut hypotube. The laser cut hypotube may be configured to extend along at least about the distal 30% of the catheter length. The laser cut hypotube may further comprise a plurality of sections, each of the plurality of sections having a distinct laser cut pattern. The distinct laser cut pattern may be associated with a flexibility of the corresponding section. The catheter may further comprise a low friction polymer coating over an exterior surface, an interior surface, or both.

### CROSS REFERENCE TO RELATED APPLICATIONS

This application claims priority to copending provisional patent application 63/432,874 filed on Dec. 15, 2022 to Wainwright et al., entitled “Catheters with Laser Cut Hypotube Scaffold for Flow Under Significant Pressure Differentials,” incorporated herein by reference.

### FIELD OF THE INVENTION

The invention relates to catheters suitable for use in a patient's vasculature, in particular the neurovasculature of the brain and peripheral vessels. The catheters generally comprise a laser cut hypotube and a sealing embedded polymer to provide a high flexibility, while at the same time providing desired constraints for significant fluid pressure differentials between the inside and outside of the catheter.

### BACKGROUND OF THE INVENTION

Aspiration catheters have found use with respect to removal of clots from vessels. Furthermore, a significant reason for ischemic injury during percutaneous procedures can be generation of emboli that block smaller distal vessels. Aspiration catheters used alone or with embolic protection device can be effective to capture emboli generated during procedures. The delivery of effective devices to the small blood vessels of the brain to remove clots and/or to capture emboli remains challenging.

Infusion catheters can be useful for various procedures. These catheters can be designed for the delivery of fluids under reasonably high pressures. As with aspiration catheters, the mechanical integrity of the catheter should be maintained under the establishment of pressure differentials with respect to positive or negative relative pressures, which can be referenced to the standard average pressure of the vessel.

The cerebral artery system is a highly branched system of blood vessels, which provide blood to the brain and are connected downstream to the interior carotid arteries. The cerebral arteries can be very circuitous. Medical treatment devices should be able to navigate along the circuitous route posed by the cerebral arteries for placement into the cerebral arteries. Other paths through various vessels can also be circuitous. Delivery of various catheters can rely on mechanical properties of the catheter.

### SUMMARY OF THE INVENTION

In a first aspect, the invention pertains to a medical catheter for transfer of fluids comprising a laser cut hypotube and an embedded polymer. The laser-cut hypotube comprises a proximal end and a distal end. The embedded polymer is flowed into the cuts of the laser-cut hypotube. The polymer can have a Shore Durometer value from about 35 A to about 76 A.

The laser cut hypotube may be configured to extend along at least about the distal 30% of the catheter length. The laser cut hypotube may further comprise a plurality of sections, each of the plurality of sections having a distinct laser cut pattern. The distinct laser cut pattern may be associated with a flexibility of the corresponding section. The catheter may further comprise a low friction polymer coating over an exterior surface, an interior surface, or both.

In a further aspect, the invention pertains to a distal access aspiration catheter comprising a laser cut hypotube, an embedded polymer tube flowed into the cuts of the laser cut hypotube, and a control wire. The laser cut hypotube comprises a distal end and a proximal end. In some embodiments, the laser cut hypotube forms a tubular element with an aspiration lumen and a connection section for interfacing with a guide catheter to complete an aspiration lumen extending through the proximal portion of the guide catheter. The control wire can comprise a flattened distal end welded or soldered to a matching configuration in the structure of the hypotube in the connection section at or near the proximal end of the laser cut hypotube.

The laser cut hypotube may comprise a plurality of sections, each of the plurality of sections having a distinct laser cut pattern. The plurality of sections may have greater flexibility in a distal direction. The plurality of sections may comprise three sections, including a proximal section and a distal section having constant laser cuts along their length and a middle section transitioning the laser cuts between the proximal section and the distal section. The distal section may have greater flexibility than the proximal section. The laser cut hypotube may comprise a flared distal end, a flared proximal end or both a flared distal end and a flared proximal end.

In another aspect, the invention pertains to an infusion balloon catheter comprising a laser cut hypotube, an embedded polymer, a balloon, and a distal tip to the balloon. The laser cut hypotube includes a port through the wall near the distal end of the hypotube. The embedded polymer is flowed into the cuts of the laser cut hypotube. The balloon generally is in fluid communication with the interior of the laser cut hypotube.

The laser cut hypotube may comprise a plurality of sections, each of the plurality of sections having a distinct laser cut pattern. The plurality of sections may comprise sections with different outer diameters and a transition section connecting two sections with different outer diameters. A more distal section may have a greater flexibility than a more proximal section. At least one section may include a spiral laser cut. The port may be formed by a gap in the embedded polymer to allow fluid flow through the laser cuts. The port may be formed through a specific hole formed though the hypotube and embedded polymer.

### BRIEF DESCRIPTION OF THE DRAWINGS

FIG. 1 is a schematic sectional view of an embodiment of a laser cut hypotube with a polymer jacket and coatings over the interior and exterior.

FIG. 2 is a schematic sectional view of an embodiment of a laser cut hypotube with a polymer liner and a polymer jacket.

FIG. 3A is an enlarged side view of an embodiment of a slightly curved widening of the distal end of a catheter.

FIG. 3B is an enlarged side view of an embodiment of a relatively constant angled widening of the distal end of a catheter.

FIG. 4 is a side view of an embodiment of a catheter with three segments.

FIG. 5 is a schematic view of an embodiment of a spiral laser cut hypotube.

FIG. 6A is an enlarged photographic view of an embodiment of a laser cut hypotube.

FIG. 6B is an enlarged photographic view of an embodiment of a laser cut hypotube.

FIG. 7 is a side view of an embodiment of an aspiration catheter.

FIG. 8 is a schematic view of an embodiment of a guide assisted aspiration catheter.

FIG. 9 is an embodiment of distal end of a catheter having an extending section that expands to a funnel.

FIG. 10 is an exploded view of an embodiment of a laser cut hypotube having an approximately constant outer diameter, a hub to be engaged with the proximal end of the catheter, and four sections noted to have distinct laser cutting patterns.

FIG. 11 is an embodiment of a guide supported aspiration catheter prototype having a laser cut hypotube divided into three distinct sections.

FIG. 12 is a fragmentary side view of an embodiment of a control structure for a guide assisted aspiration catheter showing an angled opening into the catheter lumen to provide a larger surface area opening.

FIG. 13 is a fragmentary side view of an embodiment of a control structure for a guide assisted aspiration catheter with a flared opening at the proximal end.

FIG. 14 is a fragmentary side view of an embodiment of a control structure for a guide assisted aspiration catheter with flared openings at the distal and proximal ends.

FIG. 15 is a side view of a single lumen balloon/infusion catheter with an integrated distal coil.

FIG. 16A is a side view of a tubular shaft component of the single lumen balloon/infusion catheter of FIG. 15.

FIG. 16B is a side view of a specific embodiment of the tubular shaft of FIG. 16A depicting a portion of the laser cut hypotube with polymer excluded.

FIG. 16C is a side view of a specific embodiment of the tubular shaft of FIG. 16B depicting a gap in the laser cuts around a port.

FIG. 17A is a fragmentary view of an embodiment of a connecting section of a guide assisted catheter.

FIG. 17B is a flattened view of the cutout portion of the connecting section of FIG. 15A.

FIG. 17C is a flattened view a puzzle pieced shape cutout of a connecting section.

FIG. 18 is a photographic view of a catheter bending about a mandrel.

FIG. 19 is a schematic view of an experimental system configured for real time monitoring of a simulation aspiration thrombectomy.

FIG. 20 is a chart depicting pressure and flow rate measurements for four embodiments of a catheter.

FIG. 21 is a chart depicting pressure and flow rate measurements for coated and uncoated embodiments of catheters.

### DETAILED DESCRIPTION OF THE INVENTION

Designs of a catheter incorporating a laser cut hypotube can achieve a very thin profile through the appropriate matching of materials and structures to provide a high flexibility, while at the same time providing desired constraints for significant fluid pressure differentials between the inside and outside of the catheter. For use in desired procedures, the catheters can maintain fluid containment under relatively high pressure differentials for aspiration or infusion, as described relative to vascular pressures, which provides a reference point for pressures external to the catheter. To achieve desired levels of deliverability in a patient's vasculature without compromising corresponding mechanical strength of catheter walls, a composite structure is described based on a laser-cut metal hypotube formed in a composite with intimately embedded thin polymer jacket/liner. forming a sealing polymer. Following usual use in the art, hypotube herein refers to a thin metal tube for medical device formation. Even with very thin profiles, the catheters provide good crush resistance as well as good pushability since it can be desirable to advance the catheter tip into hard to reach vessels. The catheters can be designed to flare outward to a slightly larger diameters at the distal end and in appropriate structures at a proximal end. Aspiration catheters can be full length designs or embodiments designed to have the proximal ends within a guide catheter to extend the aspiration lumen within a section of the guide catheter (distal access catheter). Various infusion catheter designs are contemplated, and specific systems are described that can provide backflow from the infusion, which can also be useful for thrombectomy. Thus, in particular, improved devices for aspiration thrombectomy procedures are described. These devices can be particularly effective for procedures in the neuro-vasculature, although the can also be desirable devices for coronary and peripheral procedures.

Providing effective aspiration or infusion catheters for small vessels in hard to reach locations in the vasculature present significant challenges. The outer diameters are limited by the vessel size and catheter maneuverability. To obtain desired degrees of aspiration or infusion, the inner diameter is desirably as large as possible consistent with the other constraints. The wall thickness is a half of the difference between the outer diameter and the inner diameter. If the wall thickness is too small, the catheter can narrow in response to negative pressures. Some commercial catheters exhibit evidence of lumen narrowing during lab testing under aspiration. Traditional catheters are made with polymer tubes often with very thin wire reinforcement. To obtain sufficient flexibility for use in many procedures, the polymers are limited with respect to their mechanical strength. Maintenance of the inner diameter during aspiration is desirable to allow for clot ingestion.

Metal tubes, generally referred to as hypotubes in a medical context, provide greater mechanical strength, but metal tubes are not sufficiently flexible for many applications. Skilled laser cutting of hypotubes has become available, and the laser cut hypotubes can be flexible. While laser cuts may not penetrate the metal wall, cuts to introduce a desired degree of flexibility pierce the metal wall. If the laser cut hypotube (LCH) is intended for fluid transfer, either aspiration or infusion, liquid would flow through the cut walls unless constrained. Placement of a polymer jacket and/or liner can increase the wall thickness, decreasing the inner diameter for a fixed outer diameter, and can reduce flexibility, reducing the effect of the laser cutting. These concerns are substantially addressed in the catheter designs described herein.

With a thinner wall design, the interior diameter along the lumen can be larger to improve aspiration to remove thrombus or to facilitate delivery of fluid. With a thinner wall design to maintain structural integrity of the tube, the overall amount of removed metal from the cutting generally would be less. Since cutting provides openings through the hypotube wall, having smaller openings through the wall can facilitate stable plugging of the openings using polymer reflow, such that the polymer functions as a sealing polymer. At the same time, good flexibility should be maintained. Maintenance of good flexibility can be facilitated by the use of a softer polymer for a polymer jacket over the hypotube. If the polymer is too soft, the polymer can be distorted by pressure differentials between the inside and the outside of the hypotube within a vessel. Suitable polymer softness generally may correlate with size and shape of openings through the hypotube wall.

A partially LCH based aspiration catheter is described in published U.S. patent application 2018/0228502A1 to Shaffer et al. (hereinafter the '502 application), entitled “Dual Lumen Hypotube Catheter,” incorporated herein by reference. The catheter in the '502 application has a distal end that is solely a wire reinforced polymer tube, comparable to a standard catheter design. The separate guidewire lumen is attached over the polymer distal part of the catheter, which creates an effective larger outer diameter. In general, the '502 patent does not address the issues resolved by the improvements described herein. Catheters asserted for use as access catheters and aspiration catheters are described in published U.S. patent application 2022/0362520A1 to Tran et al., entitled “Neurovascular Distal Access Support Catheters, Aspiration Catheters, or Device Shafts,” incorporated herein by reference. The '520 application does not described maintenance of flexibility or ability to tolerate aspiration.

The basic advantage flowing from the catheter designs relate to the material choices to provide high flexibility, good pushability and fluid containment. As taught herein these catheter improvements are discussed specifically in the context of aspiration catheters and infusion catheters for delivery of fluid under pressure behind a balloon. These specific devices can be effectively used for improved thrombectomy procedures.

The improved aspiration catheters described herein balance various aspects to provide good pushability, high flexibility, along with excellent aspiration function and kink resistance. Two primary designs are described. The first type of aspiration catheter is a design intended to have its proximal end remain exterior to the patient with a distal tip that can be delivered to a target location in a patient's vasculature. The catheter diameter can have one or more steps down in diameter toward the distal end of the catheter. For the delivery of aspiration to narrow vessels, such as neurovascular arteries or other peripheral vessels, step down catheter designs have been found to provide good aspiration performance. Such catheter designs with conventional metal reinforced polymer based catheter construction are described generally in U.S. Pat. No. 10,058,339 to Galdonik et al. (hereinafter the '339 patent), entitled “Aspiration Catheters for Thrombus Removal,” incorporated herein by reference.

A second style of aspiration catheter is a distal access style in which the catheter resides in use completely within the patient with a control structure extending from the patient. A portion of the aspiration lumen is formed by a guide catheter or the like, and the aspiration catheter can also be referred to as a guide assisted aspiration catheter. While the distal access aspiration catheter has some advantages, both aspiration catheter designs can advantageously use the structural features described herein. Full length aspiration catheter can have a constant diameter along its length, or it can have sections with different diameters. In some embodiments, a distal section of the catheter can have a narrow diameter for access into small vessels. A narrow tip aspiration catheter is described in the '339 patent cited above. These various embodiments of the full length aspiration catheter can effectively incorporate laser cut hypotube. Improved aspiration can be provided using a distal access aspiration catheter that uses a portion of the guide catheter for providing a section of aspiration lumen and collectively forming an aspiration structure. These aspiration catheters used in conjunction with a guide catheter to form the aspiration lumen are described generally in U.S. Pat. No. 10,716,915 to Ogle et al. (hereinafter the '915 patent), entitled “Catheter Systems for Applying Effective Suction in Remote Vessels and Thrombectomy Procedures Facilitated by Catheter Systems,” incorporated herein by reference. The use of the laser cut hypotube with polymer embedding introduce some design modifications for some catheter constructions.

Infusion/balloon catheters can be used to provide back flow in a vessel to assist in dislodging thrombus, which can be particularly effective in the performance of aspiration thrombectomy. Various designs for a infusion catheter can provide desirable performance. These devices are described generally in U.S. Pat. No. 11,229,445 to Ogle (hereinafter the '445 patent), entitled “Hydraulic Displacement and Removal of Thrombus Clots, and Catheters for Performing Hydraulic Displacement,” incorporated herein by reference. The LCH based catheters described herein can be adapted for various catheter components described in the '445 patent. For access into the small and circuitous vessels of the neurovasculature, a single lumen design is desirable, in which the lumen serves both as a balloon lumen and an infusion lumen. Improvements on the single lumen design of the '445 patent are described in published U.S. patent application 2023/0211134 to Ogle et al. (hereinafter the '134 application), entitled “Infusion Catheter With a Balloon Having a Single Lumen and an Internal Wire, and Used Thereof,” incorporated herein by reference. The laser cut hypotube with embedded polymer can be effectively used as catheter components for the various infusion catheter embodiments, and specific discussion below is directed to the particular embodiment of the '134 application.

The nature of a laser cut hypotube (LCH) results in a porous structure that does not provide for fluid flow through its lumen. The use of a polymer jacket and/or liner results in issues regarding the resulting thickness of the jacketed LCH, the flexibility of the hybrid structure, and the stability of the structure against pressure differentials. Applicant has discovered, that the polymer can be selected sufficiently soft to inhibit loss of desired flexibility, which is part of the motivation for use of the LCH. The polymer can be reflowed to at least partially penetrate into the cuts of the hypotube to decrease any increase in wall thickness. With polymer penetrating into the laser cuts, proper selection of the polymer can still provide for desired flexibility, even though the polymer conforms to the contortions of the LCH movements. The interpenetration of the polymer in the laser cuts also stabilizes the polymer against delamination or stretching in response to pressure differentials of the fluid inside and outside of the catheter. Proper material engineers thus provides for forming a surprisingly thin catheter structure that is stable against significant pressure differentials and maintains a high degree of flexibility.

In some embodiments, it can be desirable to form a widening at the distal end of the catheter. Due to the laser cutting, the widening can be achieved using deformation of the cut hypotube at the distal end. For aspiration embodiments, a slightly widened distal opening can provide easier entrance of the clot can be achieved with a slight fixed flaring of the catheter body or an extendable section that can be delivered in a low profile. Since the aspiration force is significant, even a slight enlargement of the diameter can redirect some of the aspiration force into compressive force to help get the clot into the catheter lumen without significantly impacting the catheter delivery. A slight flaring may also help the catheter to resist deformation due to pressure of the clot against the opening during ingestion of a clot into the catheter.

As described herein, the improved catheter structures can be effectively used to form aspiration catheters as well as infusion/balloon catheters. The catheter structures have very desirable properties can make them suitable for a range of catheter uses that extend beyond the specific devices described herein. In addition, while the designs are motivated by the challenges of the neurovasculature with its narrow vessels and tortuous path, the catheter structures can effectively be adapted for catheters for cardiac procedures, various peripheral vessels or other medical applications. Due to the availability of commercial LCH processing, these catheter structures can be provided in cost effective commercial embodiments.

The basic materials and construction of the catheter structures based on laser cut hypotubes with embedded polymer are described next in detail Then, some specific catheter embodiments are described in detail. Aspiration results are then presented from bench studies of a distal access aspiration catheter design to demonstrate the function of the catheter under significant vacuum conditions.

### Structures With Laser Cut Hypotubes With Embedded Polymer

To achieve the desired levels of flexibility while maintaining good deliverability and aspiration stability and performance, the catheter construction incorporates several features. The core of the structure comprises a laser cut hypotube with appropriately selected dimensions and cut properties, as described below. A polymer tube can be placed over the laser cut hypotube (LCH) as a jacket or inside the LCH as a liner. The polymer tube generally has a thickness less than the thickness of the LCH, and the polymer tube can be selected to have a soft material indicated by an appropriate Shore Durometer value. When the polymer tube is reflowed by heating the polymer to secure it to the LCH, the polymer can partially flow into the cuts of the LCH, which is demonstrated to provide good adhesion in the exemplified embodiments. In some embodiments, the polymer can be applied as a liquid and cured to form the embedded sealing polymer. Due to the relatively low Shore Durometer hardness, the reflowed polymer does not significantly adversely effect the catheter flexibility even though it is at least partially filling in the cuts. With the small profiles of the laser cuts and the reasonably balanced polymer tube parameters, the catheter is amenable to fluid flow of the catheter lumen, and aspiration results are presented below. Lubricious coatings can be applied internally and/or externally to further improve catheter performance with only very little change in diameters.

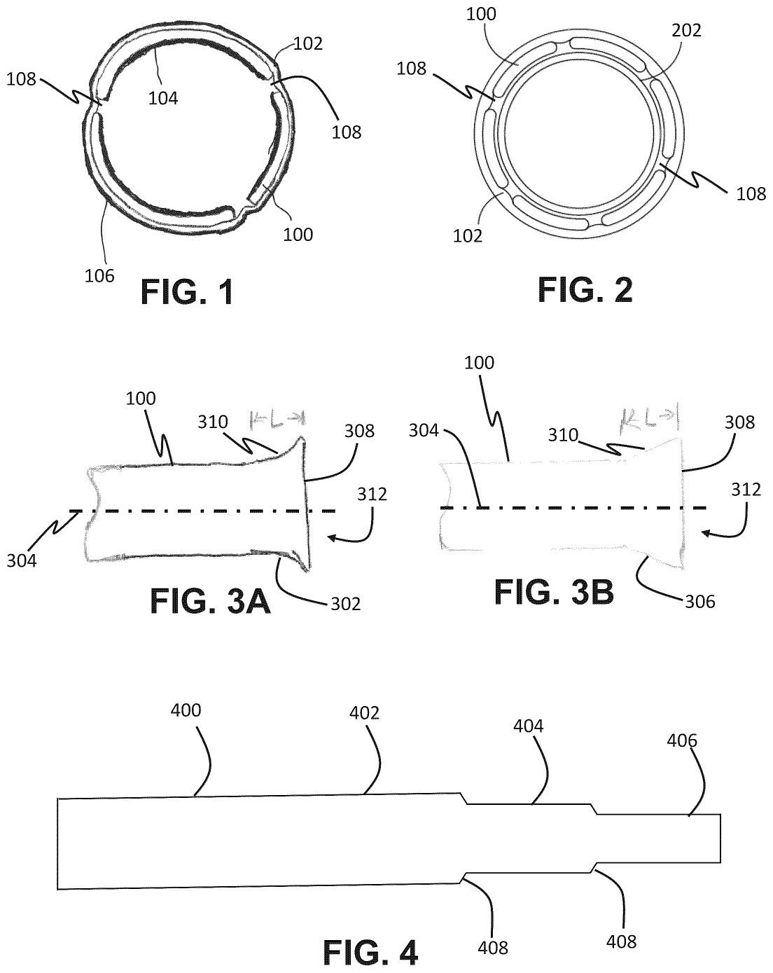

Referring to FIG. 1, a schematic sectional view is shown with segments of the laser cut hypotube 100, with a polymer jacket 102 and coatings over interior 104 and exterior 106. The coatings 104, 106 are optional, but they may improve performance. Polymer jacket 102 reflows to at least partially fill cuts 108 through laser cut hypotube 100. In some embodiments, the reflow can essentially fill cuts 108. The reflow helps to secure polymer jacket 102 to laser cut hypotube 100 to resist delamination of the polymer jack in response to pressure differentials between the interior and exterior of the catheter.

Referring to FIG. 2, a schematic sectional view is shown for an embodiment with both a polymer liner 202 and a polymer jacket 102. The catheter can similarly be formed with just the polymer liner 202, and the interior and/or exterior can have a coating, such as coatings described below. As shown schematically in these figures, the polymer jacket 102 and/or liner 202 can migrate at least in part into holes 108 of laser cut hyoptube 100 during processing, which generally comprises a heating reflow process that also bonds the polymer structures to laser cut hypotube 100.

As noted above, a widening at the distal end of the catheter can provide easier entrance of a clot based on a slight fixed flaring of the catheter body. As described below, alternatively an extendable section can be used that is delivered in a low profile. Since the aspiration force is significant, even a slight enlargement of the diameter at the distal opening can redirect some of the aspiration force into compressive force to help get the clot into the catheter lumen without significantly impacting the catheter delivery. Generally, the enlarged section 310 can be curved 302 along the axis 304 of the catheter 100 (see FIG. 3A) or have a relatively constant angle 306 (see FIG. 3B), which can have the feature of a relatively constant force on the clot over the section. The slight flare 310 of the distal tip 308 can be formed by stretching the distal opening 312 of the hypotube 100 prior to application of the polymer jacket and any polymer coatings. The flaring 310 should be slight to avoid any complications for the catheter delivery due to the larger diameter distal tip 308. The resulting funnel shape of the distal tip 308 can have a linear growth of the diameter with a straight angled edge 306 and a relatively discontinuous edge where the flare transitions to a constant diameter section, or the flaring section 310 can have a nonlinear shape 302 as desired. The distal outer diameter can be, for example, up to about 10% larger than the average outer diameter of the adjacent proximal catheter section, and in some embodiments up to about 8% larger and in other embodiments from about 1% to about 5% larger. The length (L in FIGS. 3A and 3B) of the enlarged segment balances the amount of compressive force, which is greater for a steeper angle (sine of the angle), versus the length over which the angle is applied. The length of the flared section along the length of the catheter can range from about 0.1 mm or less to a gradual transition over the distal section of the catheter. In some embodiments, the flared section has a length along the axis of the catheter of no more than about 1 cm and in some embodiments no more than about 5 mm. The extending process to form the slightly enlarged diameter may naturally determine the extent along the length of the catheter of the enlarged diameter section if the distal end is directly extended by stretching the metal. A person of ordinary skill in the art will recognize that additional ranges of extended diameter sections within the explicit ranges above are contemplated and are within the present disclosure. The expanded section may or may not be covered with the polymer jacket, liner, and/or coatings. Catheters with an extended diameter distal tip can be applied for other applications if desired distinct from use as an aspiration catheter.

The combination of materials provides desirable performance with a very thin wall thickness. Generally, the catheter has an average wall thickness from about 0.002 inches (in) to about 0.010 in, in some embodiments from about 0.0025 inches (in) to about 0.0085 in, in further embodiments from about 0.00275 in to about 0.008 in and on other embodiments from about 0.003 in to about 0.0075 in. (One inch (in.) is equal to 25.4 mm.) In some embodiments, the wall thickness can be uniform over the length, and in some embodiments, the thickness of the catheter varies by no more than about 10% from the average at any point. The LCH generally has an outer diameter selected based on the particular target application and an inner diameter based on the wall thickness after the outer diameter is selected. The wall thickness of the LCH can fall within the same ranges as the catheter wall thicknesses provided above. The difference between the catheter wall thickness and the LCH wall thickness can generally range from about 0.0005 in. to about 0.006, in. in other embodiments from about 0.0005 in. to about 0.00125 in and in further embodiments from about 0.00125 in to about 0.060 in (+0.0005 to 0.001 in). The ranges are indicated as starting at 0.0005, and generally at least a lubricious coating having a thin layer to reduce friction, although the average thickness of the coating can be in the range of the variation in wall thickness. A person of ordinary skill in the art will recognize that additional ranges of wall thicknesses within the explicit ranges above are contemplated and are within the present disclosure.

For reaching small cerebral arteries, the outer diameter generally ranges from about 1 French (Fr) to about 9 Fr, and in some embodiments from about 3 Fr to about 6.5 Fr. In some embodiments, different outer diameter catheters can be distributed such that a medical professional can select the desired size for a particular procedure. The inner diameter is equal to the outer diameter minus twice the wall thickness. A catheter, such as an aspiration catheter, can have different sections with different outer diameters to balance performance, such as aspiration performance, and ability to reach target narrow vessels. A catheter can have two, three or more than three segments with more distal segments having a narrower diameter, with appropriate transition zones connecting the segments. An embodiment with three segments 402, 404, 406 is shown in FIG. 4. In some embodiments, the outer diameter differences between adjacent segments can generally be Fr/3, Fr/2, 1 Fr, 1.5 Fr, 2 Fr, or ranges based on these values, such as Fr/3 to 1.5 Fr, although other values can also be appropriate. In embodiments in which the wall thickness remains constant, the inner diameters would have roughly the same differences as the outer diameters. A person of ordinary skill in the art will recognize that additional ranges of wall thicknesses and diameters within the specific ranges above are contemplated and are within the present disclosure.

For a segmented catheter 400, the laser cutting parameters can be different in the different segments 402, 404, 406, generally, to have more flexibility in the distal segments. The laser cutting of each segment 402, 404, 406 can be selected within the parameter ranges indicated below. As noted below, the laser cutting can also be varied along a segment. To form a segmented structure, a hypotube can be initially formed with the different diameters in the separate segments, and then the segmented hypotube can be laser cut. Alternatively, separate segments can be formed and laser cut, and the separate segments can be welded together or connected with polymer segments to secure the laser cut hypotube segments, in which the polymer segments can be secured to the laser cut hypotube by reflowing the polymer, using an adhesive, combinations thereof, or other suitable fastening approach.

### Catheter Structures—Laser Cut Hypotubes With Sealing Polymer

The overall structure of the catheter can be assembled on the laser cut hypotube. In general, the polymer seal component can be introduced initially as a jacket, a liner, or applied as a fluid. In some embodiments, a polymer jacket is placed over the exterior of the LCH. If desired, one or more additional layers can be placed over the polymer jacket, such as a heat shrink layer or a lubricious coating. An inner polymer liner or coating may or may not be used if a polymer jacket is used. Examples are presented for an aspiration catheter embodiment with a polymer jacket, and with or without an inner lubricious coating showing some decrease in aspiration times based on the coating. A polymer jacket or liner can be thermally flowed to at least partially filling into the gaps in the hypotube from the laser cutting. The reflow can involve heating just above the polymer softening point, which can depend on the particular polymer. The polymer jacket can initially have a thickness from about 60% to about 150% of the LCH thickness, in further embodiments from about 70% to about 140% and in other embodiments form about 75% to about 130% of the LCH thickness. Following reflow, the polymer jacket can be essentially fully embedded in the cuts of the LCH or a fraction of the polymer thickness can be maintained above and/or below the surface of the LCH. The overall thickness of the catheter wall falls within the ranges above with respect to the overall thickness relative to the LCH thickness. A person of ordinary skill in the art will recognize that additional ranges of relative polymer jacket thicknesses within the explicit ranges above are contemplated and are within the present disclosure.

In one embodiment, a biocompatible polyurethane or other viscous polymer could be brushed or sprayed onto a removable mandrel (silver core, copper core, PTFE, or other). This could either be cured before or after sliding the LCH over the polyurethane or other polymer coating. If cured before sliding LCH on, then the polymer could be ground or skived to ensure concentricity. A taper on mandrel could be used to help slide LCH on and even a sharper edge on LCH to enable shearing while loading LCH. A layer of a biocompatible polyurethane or other low Shore Durometer value polymer could be brushed or sprayed onto the LCH and reflowed if necessary and then cured. Suitable polymers for application in this way include, for example, polyurethanes from Biomerics (e.g., Quadraflex ARE™, Elast-Eon™ and Quadrasil ARCS™) and polyurethanes from DSM (Bionate® and Elastane®). In some embodiments, the reflow temperatures are on the order of 200° ° C., and generally in some embodiments from about 60° C. to about 250° C. The reflow would create better adhesion between the inner and outer layers. The OD could then be skived or ground to the correct dimension. After this securing of the outer layer, the part can be thoroughly cleaned to remove particulate, and then the OD and/or ID can be coated with a lubricious coating. The benefit of this construction process is that softer polymers could be used because it is applied as a liquid. Further, there can be good adhesion between inner and outer layers. Further, because a lubricious coating could be applied to both ID and OD, a tacky but softer polymer could be chosen to interface directly with the LCH. Coatings, such as hydrophilic coatings are described further below and can be thin.

The laser cuts provide significant improvements in flexibility relative to an uncut hypotube, while maintaining significant crush resistance and pushability. In some embodiments, the laser cuts extend virtually the entire length of the catheter tube with small uncut segments at the ends for stability of the cut sections and avoid fraying. The small uncut sections can be from about 0.0020 in to about 0.1 in and in further embodiments from about 0.0030 in to about 0.060 in. The proximal and distal uncut sections may not have the same dimensions. For use as aspiration catheters, two catheter designs are considered, a full length and a short, guide supported version. These catheter designs can be adapted for other applications. In particular, the full length catheter can be adapted as an outer tubular component of an infusion/balloon catheter discussed further below. The proximal end of the full length catheter generally fits into a hub, and while this segment to be fit into a hub may not be cut, cutting this section according to the values above can provide some strain relief. So there would be no particular impetus to relax the dimensions of the uncut section of the full length catheter. A larger uncut section could provide increased pushability, but the essentially fully cut hypotube can provide good pushability while not sacrificing flexibility, and the parameters of the laser cuts can be varied according to sections to balance these factors along the length of the catheters. At the proximal end of the distal access (guide supported) catheter version, the hypotube can be differently cut to provide for attachment of a control structure and optionally to modify the opening into the lumen to facilitate insertion of instruments. A differently cut section can be the same overall length as the uncut sections mentioned above. Some specific options for this section are described below. A person of ordinary skill in the art will recognize that additional ranges of uncut dimensions within the explicit ranges above are contemplated and are within the present disclosure.

While the catheter structure provides some radiopacity, it can be generally desirable to include at least one radiopaque marker, such as a band, for example, near the distal end of the catheter. A marker band can comprise a particularly radiopaque metal, such as platinum-iridium alloy, tantalum, tungsten, gold, platinum-tungsten alloy composites thereof, or mixtures thereof. It has been found that a marker band can itself be laser cut to improve flexibility of the resulting structure around the marker band. For improved attachment of a marker band, a marker band can be placed under the polymer jacket. To avoid a more significant change of flexibility at the marker band, a marker ban can itself be laser cut with a similar or different cut pattern as the hypotube, or it can be formed from loosely braided metal wire. Laser cut marker bands are described further in copending U.S. patent application 17/18.209,335 to Wainwright et al., entitled “Embolic Protection Device Designed in Particular for Tortuous Blood Vessels, Especially Cerebral Vessels,” incorporated herein by reference. The markerband (MB) could be welded or otherwise adhered to the LCH. It could then be ground to ensure uniform OD. The MB could be laser cut as part of the LCH processing or laser cut before or after attaching to the LCH. The MB could also be stretched similar to the distal end of the LCH to create better flexibility. A soft radiopaque material could be used such as Ta or Pt or a stiffer material such as W, PtW, or PtIr. Super elastic materials could also be used for distal end including, for example, NiTi, NiTiCr, (Fort Wayne Metals) Ti-beta, or potentially more beneficial a radiopaque and super elastic material such as MoRe alloy or W26Re alloy. If Superealstic, then this could help with flaring of distal end to improve aspiration force. In combination with the polyeurethane described above, this could create a cohesive, durable, flexible, radiopaque and lubrcious funnel. Alternatively, a radiopaque coil or braid could be welded or otherwise attached to the LCH.

As noted above, the laser cuts on the LCH should have appropriately small dimensions to provide for fluid pressures without compromising a soft polymer jacket. An intermittent spiral cut can be efficiently cut, and these can be essentially equivalent to corresponding circumferential cuts. A spiral cut can be characterized by angular extent of a cut section, angular length of an uncut section, cuts per revolution and pitch. Depending on the selection of these parameters, the uncut sections may or may not be aligned along the length of the hypotube. The width of the laser cut, referred to as the Kerf, can be 0.001 in ±0.0005 in. If the state of the art of laser cutting evolves further, corresponding adjustments can be made accordingly.

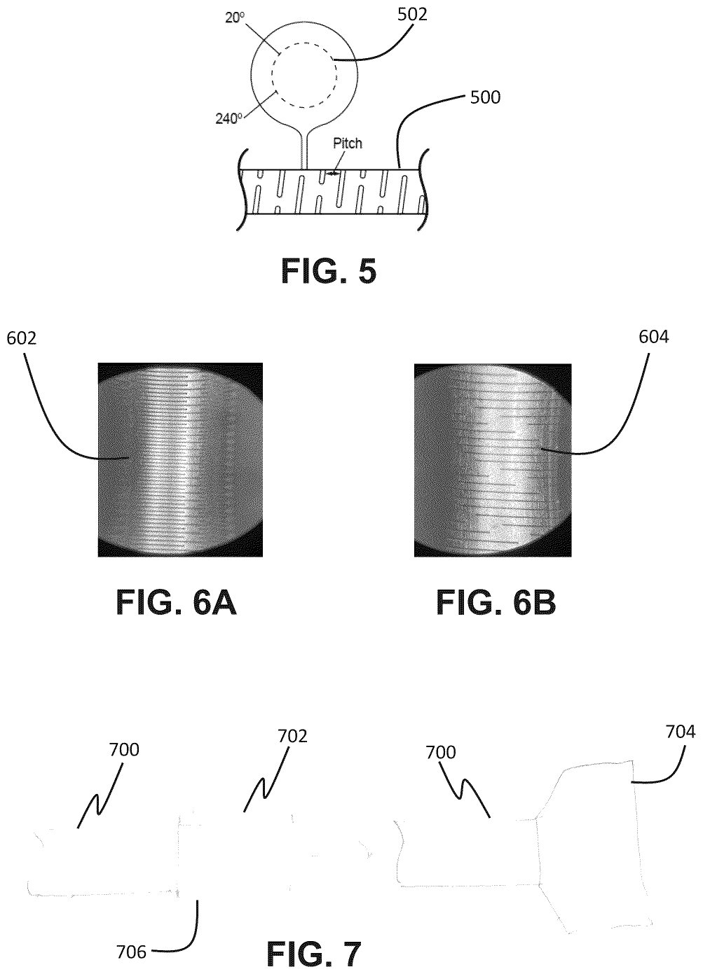

A schematic view of a spiral laser cut hypotube 500 is shown in FIG. 5 with pitch noted along with an enlarged photographic views in FIGS. 6A (1.5 CPR) 602 and 6B (1.85 CPR) 604. The insert in FIG. 5 depicts along a spiral cut 502 with 1.5 CPR, 240° cut and 20° uncut angle. To achieve the desired flexibility of the catheter, the pitch can range from about 0.0030 in to about 0.015 in, and different sections can have adjusted pitch to vary the flexibility to balance pushability and flexibility. The pitch is the edge-to-edge distance between adjacent cuts. A smaller pitch generally is associated with greater flexibility. The cuts per revolution (CPR) can generally range from about 1.2 to about 5.0 and in some embodiments from about 1.4 to about 2.5. The angle of a cut can correspondingly range from about 60° to about 260° and in some embodiments from about 120° to about 245°, and the range of uncut angle can be from about 10° to about 60°. A person of ordinary skill in the art will recognize that additional ranges within the explicit ranges above are contemplated and are within the present disclosure. To transition from a more flexible distal end to a less flexible proximal end, the pitch and/or the CPR (with corresponding decrease in cut angle and/or increase in uncut angle) can increase to decrease flexibility. In some embodiments, the catheter can have two or more fixed parameter cut regions, such as 3, 4 or more than 4, connected by a transition region where the parameters gradually change from one set of parameters to another. The transitions could be gradual and the metal LCH could be deformed on a tapered mandrel and heat-set. In alternative or additional embodiments, essentially abrupt changes in LCH parameters can take place at adjacent fixed parameter cut regions. In this embodiment, the LCH could have both radial flexible cuts as well as regions to enable expansion. The radial regions and expansion could be within the same region or separate. Alternatively, multiple hypotube diameters could be used to create a stepped catheter. The individual hypotubes could be laser cut prior to or post welding, soldering, sintering together or other joining methods a person of ordinary skill in the art will recognize. The resulting stepped structure 408 forms a catheter, such as shown in FIG. 4. The stepped approach may be beneficial for a lower cost of goods. A person of ordinary skill in the art will recognize that additional ranges within the explicit ranges above are contemplated and are within the present disclosure.

The properties of the laser cuts, the thickness of the hypotube, the hypotube metal (e.g., stainless steel, titanium, nitinol), the polymer jacket properties, other components contribute the resulting flexibility of the catheter. Specific examples of cut patterns are presented below for prototype catheters, one full length aspiration catheter and one guide supported aspiration catheter. Aspiration results are presented for the guide supported aspiration catheter prototype. Laser cut hypotubes according to appropriate parameters are available from Resonetics.

The distal end of the LCH can additionally or alternatively be stretched along the axis of the tube, prior to association with a polymer jacket, to increase the flexibility of the distal end of the LCH. The stretching can be performed by holding the tube and pulling at the distal end. For prototyping, this stretching was performed by hand, but for commercial consistency, this can be performed with a fixture. The small change in length can effectively keep the diameter unchanged. The configurations of the cuts can be slightly altered by the stretching. The laser cuts can be adjusted to cut a more diamond-like shape, or other shape corresponding to further removed portions of metal with greater flexibility, rather than a line of the laser beam width, for example, to form cuts of similar shape as formed by the stretch, which can be used as an alternative to the stretching. The stretched (105% to 400% of original length) or alternatively cut section of the LCH can extend from less than 2 inches to the last inch or less of the hypotube. Laser cut hypotubes formed from nitinol or similar metals can be stretched greater amounts generally. The stretched region can elastically rebound a significant fraction of the stretch, but the length remains somewhat distended. Stretching generally is performed prior to application of a polymer jacket, polymer liner and/or any coatings, to avoid damaging the polymer components or delaminating components from each other. While any dimensional changes can be minor, the resulting flexibility can provide a desirable added flexibility. A person of ordinary skill in the art will recognize that additional ranges of stretch length within the explicit range above is contemplated and within the present disclosure.

A polymer jacket can comprise biocompatible polymer materials with desired mechanical properties. It has been discovered that polymers with appropriately low Shore Durometer values can be bonded to the LCH without significantly impacting the flexibility while maintaining its integrity under high vacuum conditions. Generally, the polymer jacket can have a Shore Durometer value from about 25 A to about 76 A, in some embodiments from about 35 A to about 65 A, and in further embodiments from about 42 A to about 58 A. Suitable polymers include, for example, NeuSoft™ thermoplastic polyurethane from Avient, Pellethane® thermoplastic polyurethanes from Lubrizol, or Tecoflex™ thermoplastic polyurethanes from Lubrizol. The thickness of the polymer jacket can be from about 0.0008 in to about 0.006 in, and in some embodiments from about 0.0025 in to about 0.005 in. The polymer can be extruded and placed over the LCH. The extruded polymer tube can then be placed over the LCH and reflowed through heating to the softening point to induce bonding to the LCH with some reflowed polymer generally extending into the laser cuts through the hypotube. A person of ordinary skill in the art will recognize that additional ranges of Shore Durometer values and thicknesses within the explicit ranges above are contemplated and are within the present disclosure.

With proper attention to the thickness and softness considerations, a polymer jacket 102 the LCH 100 can have one or multiple layers, an optional liner 202 can be used, and optional coatings 104, 106 can be applied to the inner and/or outer surface to improve lubricating properties. These are depicted schematically in FIGS. 1 and 2. In particular, a coating 104, 106 can be applied to the inner surface and/or the outer surface without impacting significantly the other properties of the catheter. Along the inner surface, a coating can facilitate removal of a clot by casing its passage down the catheter once the clot enters the distal end, and a coating on the outer surface can case delivery of the catheter. Suitable coatings include, for example, Hydak® hydrophilic coatings from Biocoat, Coatings2Go™, or any biocompatible hydrophilic coating. These coatings can be applied and cured, for example, either using heat or UV light. The thickness of the coating can be roughly a few microns thick, so a coating can add a negligible amount to the overall thickness. Prototypes with and without an interior coating are described below.

Using catheter construction as described herein, the resulting structures can be used to deliver aspiration at relatively high negative pressures into vessels without delaminating or otherwise destroying the layers of the catheter wall. In other embodiments described below, the catheter can be used to deliver moderately high fluid pressures within a blood vessel without bursting the polymer jacket to avoid leaking of fluid through the wall. In these embodiments, the composite structure can comprise a low durometer polymer jacket and/or liner so that the polymer structure does not inhibit the flexibility significantly while still providing constraints of the fluid flowing through the catheter. The laser cut hypotube inhibits collapse during aspiration or ballooning of the catheter during pressure delivery, and the polymer jacket has sufficient burst strength to avoid tearing during delivery of positive or negative pressure. By selecting the thickness of the components to be thin individually, an overall thickness can be made desirably small for use in small blood vessels, but the joining of the components is performed to essentially eliminate the possibility of separation of the components even under significantly large pressure differentials.

The design of the catheter structure balances several issues. The catheter can be deliverable into difficult to reach vessels, such as cerebral arteries in the brain. To make this possible, the catheters should be pushable and highly flexible. At the same time, it is desirable for the catheters to have a small outer diameter and a larger inner diameter, which corresponds to a thin wall thickness. Designs to achieve these objectives are described herein. The laser cut pattern can be made systematic, as described below, but in this context, the cut pattern can be different near the distal end relative to other portions of the catheter to provide increased flexibility near the distal end.

In additional or alternative embodiments, the distal end of the catheter 700 can have an extending section 702 that expands into a larger funnel 704. (See FIG. 7.) Generally, the extended funnel 704 can be delivered in a low profile configuration 706 (FIG. 7, left view) and extended once at the desired location (FIG. 7, right view), where the transition is noted by an arrow in FIG. 7. The extended funnel 704 can be extended out to the vessel wall, if desired, or to a lesser extent. An extending funnel 702 generally would not be covered in the polymer jacket, but if desired, a stretchable fabric cover, such as a woven fabric, an expanded polytetrafluoroethylene, or a Bioweb™ (Zeus Inc.), can be carried on and extending with the funnel section to help to direct flow into the catheter. A self-extending funnel element for an aspiration catheter is described in published U.S. patent application 2022/0000500 to Arad Hadar et al., entitled “A Thrombectomy System and Methods of Extracting a Thrombus from a Thrombus Site in as Blood Vessel of a Patient,” incorporated herein by reference. This device is formed from a shape memory metal and extends upon delivery from a catheter. Actuatable elements are described further below that can be adapted for a funnel section.

For an extendable section 702, a material change may take place at the extendable section 702. If there is a polymer covering of some kind, this generally has a different nature than the polymer jacket over the remainder of the structure. To achieve desired control of the extension of the diameter, the frame material also generally changes. If the structure is self-actuating, a sheath or the like generally covers the tip until retracted once in position. Alternatively, resistive heating can be applied to stimulate a structural change, and electrical wires, appropriately electrically insulated, can be tracked from the proximal end to the shape changing distal tip to control the extension. A thermal actuating element can be formed from nickel titanium ally (NITINOL) shape memory metal that provides for both resistive heating and thermal shape changes. The Nitinol element can be formed with a shape memory of the extended configuration and then placed in the reduced diameter configuration for delivery. In some embodiments, a coil of the thermal actuator can comprise the resistive heating alloy within a polymer coating, although it may be desirable to not coat the alloy to provide for faster cooling upon discontinuing electrical current. Stopping the current flow and cooling the element may result in a partial return of the un-extended configuration to provide for easier removal. Extending structures are described further below in the context of interface sections, which can be applicable for these sections.

As described in the '339 patent, the combination of a wider proximal section of aspiration lumen combined with a narrow distal tip provides overall improved aspiration function. These principles are carried into the catheters of the '915 patent. While the narrower distal tip can be incorporated into an effective aspiration system, clots still need to enter the distal opening for removal. And the catheter should have sufficient pushability yet have a high flexibility to navigate the tip to the desired location for the procedure. Recent bench studies have shown that catheters constructed as described in the '915 patent have excellent aspiration abilities, but for some clots the majority of the time during clot removal can be spent with the clot corked at the distal opening gradually being brought into the catheter. See, copending U.S. patent application Ser. No. 18/514,518 to Wainwright et al. (hereinafter the '518 application), entitled “Rapid Clot Removal Using Aspiration Catheter With Aspiration Guided by Monitoring Aspiration Performance,” incorporated herein by reference. Depending on the clot structure, the clot may need to be stretched or compressed to fit through the distal opening, and aspiration forces facilitate this process. In some embodiments, the catheters herein have extended tip diameters to facilitate entry of the clots in combination with the adjacent narrow tips to provide for desirably strong aspiration. Even a small radial expansion at the tip can be beneficial for clot ingestion into the catheter lumen since the aspiration forces translate into a small compressive force against the clot.

Some general embodiments of catheters are discussed in some detail next. In this context, a full length aspiration catheter, a distal extension (guide supported) aspiration catheter and a infusion/balloon catheter are presented.

Referring to FIG. 8, a side view is shown of an aspiration catheter 800 designed with full length. The proximal end 802 has a hub 804, which is intended to remain outside of the patient. The hub 804 can be a standard catheter hub, which has a connector 806 such as a luer connector for attachment to fittings having various valves, manifolds and the like, as appropriate. As shown, the catheter 800 has a tubular element 810 with a proximal segment 812 and an optional narrower distal segment 808, and a prototype full length catheter is shown below with a straight tube with constant diameter. Tubular element 810 can be formed using polymer sealed laser cut hypotube as discussed in detail above, and this discussion of the laser cut hypotube features and the embedded polymer are as if they are copied here word for word to support this direct application of these features. If an optional narrower distal segment 808 is included, tubular element 810 can be formed as described above in the context of FIG. 4. Distal segment 808 may include a marker band 814. As discussed above, the marker band may be configured for minimal impact on flexibility. For example, the marker band may be a laser cut marker band 816 or a woven marker band 818.

A distal access aspiration catheter is designed to interface with a guide catheter to form an aspiration lumen that spans the distal access aspiration catheter and part of the guide lumen. The resulting aspiration catheter system can provide excellent aspiration performance for thrombectomy procedures. The construction of the distal access aspiration catheter with polymer embedded laser cut hypotube can provide improved deliverability, resistance to kinking or collapse from aspiration pressures, while maintaining excellent aspiration performance. The laser cut hypotube components impose design changes for implementing the overall structure with properly connected components.

The distal access (guide supported) aspiration catheter design described herein is used with the entire catheter (apart from a control wire) residing within the patient and configured with a guide catheter forming a portion of an aspiration lumen, or the distal access catheter can be used as an access lumen if the catheter were just used for device delivery. The distal access aspiration catheter is a guide supported catheter and has a control structure, such as a wire, extending proximally from the catheter and exiting the patient to provide for positioning the catheter. A distal access aspiration catheter can have an interface or connection section that interfaces with the inner surface of the guide catheter to form an aspiration lumen that is effectively sealed from flow into and out from the distal opening of the guide catheter without flowing through the interior of the aspiration catheter. These aspiration catheters used in conjunction with a guide catheter to form the aspiration lumen are described generally in the '915 patent cited above. As described herein, the construction of the aspiration catheter on a LCH core can provide for excellent performance with desirable construction features.

Referring to FIG. 9, an embodiment of a distal access (guide assisted) aspiration catheter 900 is shown schematically. A control element 902, e.g., a control wire, extends from the proximal end of the catheter. An insert in FIG. 9 shows a portion at the proximal end 904 of the catheter cut and flattened to show a rectangular cut 906 for attachment of the control wire 902, which can be flattened for attachment, and structure indicating an angled cut opening 908 to provide an opening with a greater surface area. Alternative cut out shaped for control wire attachment are described below. This construction can allow for strong attachment of the control wire without greatly increasing the catheter profile. The control wire 902 can be welded or otherwise secured to the laser cut hypotube 910, and a polymer jacket or the like over the laser cut hypotube 910 may be slit or molded around the attachment to provide secure attachment with less strain. To form a connection section, the polymer jacket near the distal end of the distal access aspiration catheter 900 can be thicker to provide for interaction with the inner lumen of a guide catheter. The proximal end 904 of the catheter 900 has an interface section 912 that engages with the inner wall of a guide catheter or the like. The lumen of the distal access (guide assisted) aspiration catheter 900 can have a constant inner diameter, or it can have one or more narrowing sections toward the distal end 914.

The length of the catheter depends on whether or not the catheter is a full length catheter or a design that uses a section of the guide catheter for forming the lumen, e.g., aspiration lumen, such as based on the MIVI Neuroscience, Q™ Catheter design, which can be referred to as a distal access or guide supported (aspiration) catheter, or which may or may not be used as an aspiration catheter. A distal access aspiration catheter can have a length from about 3 cm to about 60 cm, in some embodiments from about 5 cm to about 55 cm and in further embodiments from about 8 cm to about 50 cm. The length of full length aspiration catheters can be selected based on the desired target and the selected delivery path. For example, for delivering a catheter to cerebral arteries for acute stroke treatment, the catheter can be introduced into a femoral artery for delivery up the aorta to the branches into the carotid arteries or through an artery in the arm to the brachiocephalic artery for directing into the right or left carotid artery, which could be performed with a shorter catheter. Generally, the length of a full length aspiration catheter can be from about 45 cm to about 200 cm and in further embodiments from about 60 cm to about 180 cm. A person of ordinary skill in the art will recognize that additional ranges of dimensions within the explicit ranges above are contemplated and are within the present disclosure.

A guide catheter is generally used regardless of the configuration of a treatment catheter. The guide catheter can have an outer diameter (D) from about 5.5 Fr (1.667 mm diameter) to about 10 Fr (3.333 mm diameter), in further embodiments from about 6 Fr (1.833 mm diameter) to about 9 Fr (3 mm diameter), and in some embodiments from about 6.25 Fr (2 mm diameter) to about 8.5 Fr (2.833 mm diameter). The guide catheter measurement are generally referenced to the outer diameter, and the inner diameter is less than the outer diameter by twice the wall thickness. In general, the inner diameter of the main portion of shaft 164 (d₁) can range from about 0.8 mm to about 3.175 mm, in further embodiments from about 0.9 mm to about 2.85 mm and in additional embodiments from about 1.00 mm to about 2.7 mm. The reduction in inner diameter of distal section 188 (d₂) relative to the inner diameter of an engagement section of shaft 164 (d₁) can be from about 0.034 mm (0.00134 in) to about 0.25 mm (0.0098 in) and in further embodiments from about 0.05 mm (0.002 in) to about 0.20 mm (0.0079 in). The length of the guide catheter shaft can be from about 30 cm to about 150 cm, in further embodiments from about 35 cm to about 130 cm and in additional embodiments from about 40 cm to about 120 cm and is generally selected to be suitable for the corresponding procedure. In some embodiments, distal section 188 can have a length (L_{d}) from about 1 mm to about 50 mm, in further embodiments from about 1.5 mm to about 25 mm, and in other embodiments from about 2 mm to about 20 mm. A person of ordinary skill in the art will recognize that additional ranges of dimensions within the explicit ranges above are contemplated and are within the present disclosure.

As noted above, the catheter can have a flared distal end to form a slightly larger diameter at the opening. For a distal access (guide supported) catheter, the proximal opening can additionally or alternatively be similarly flared to expand the diameter to provide for easier entrance into the lumen. The LCH can be distended to form these flared openings. If one or both ends of the LCH are stretched to form a flared opening, the LCH generally can be appropriately altered prior to applying a polymer jacket, polymer liner and/or coatings to reduce the chance of damaging the polymer component or delaminating the elements from each other. The polymer components can be appropriately fit to the flared LCH for reflow and/or curing. The materials described herein should be amenable to forming these slightly altered forms. The extendable distal ends are described further below.

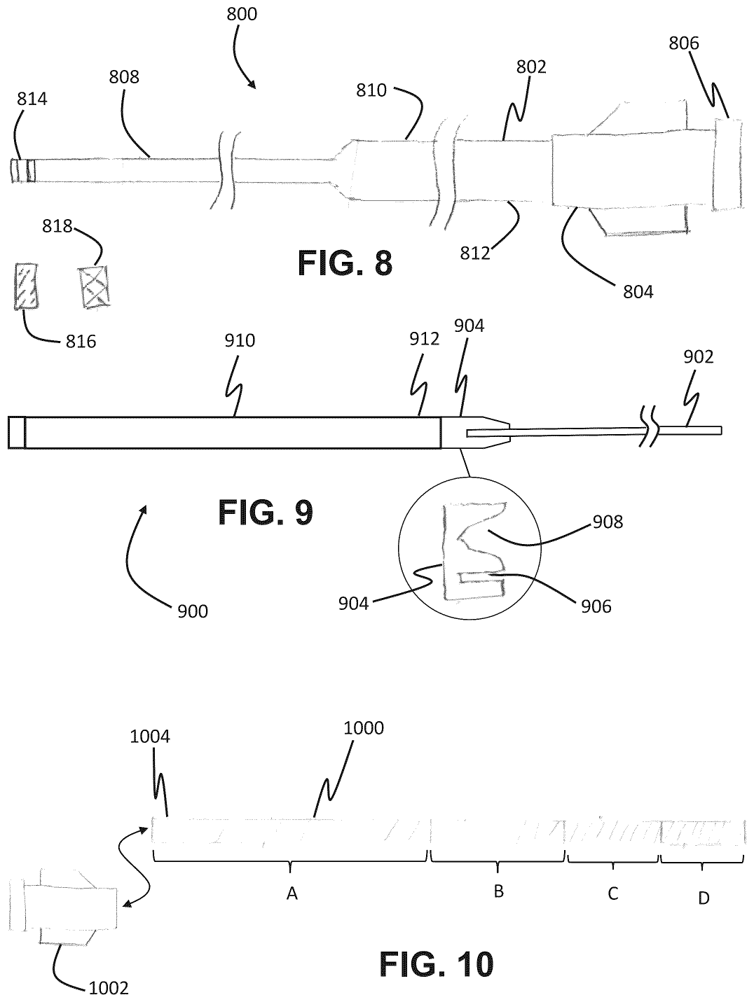

With respect to a full length catheter, the proximal end designed to extend from the patient generally has a hub to allow for secure and sealed connections to the catheter lumen. Hubs are ubiquitous in the catheter space. While specialized hubs can be made and used, hubs can be obtained commercially and assembled onto the catheter. Attachment of the hub involves fitting the opening of the hub over the end of the catheter and bonding the hub onto the catheter proximal end, for example, using heat bonding, adhesive bonding, other suitable approaches and combination thereof. The hub generally has a connector such as one portion of a standard Luer connector for making a convenient and leak proof attachment to proximal fittings. An exploded view is shown in FIG. 10 of a LCH based catheter 1000 having an approximately constant outer diameter, a hub 1002 to be engaged with the proximal end 1004 of the catheter 1000, and four sections A, B, C, D noted to have distinct laser cutting patterns to alter the flexibility along the catheter length as noted above. Specifically, the flexibility is generally increased toward the distal end to improve guiding through vessels with less impact on pushability. Thus, for a spiral cut, the amount of metal removed generally increases toward the proximal end, which can be accomplished by adjusting the various spiral cut parameters. One particular set of parameters are provided below for a test prototype. The polymer jacket, polymer liner or the like, as well as any optional coatings on the exterior and/or interior, can be secured and embedded into the laser cut pattern prior to attachment to the hub.

As noted above, the distal portion of the catheter can have a narrower diameter to provide access into narrower vessels. Sec '339 patent cited above. The use of multidiameter LCH based catheters are described above. These can be readily adapted to form an aspiration catheter according to the '339 patent and as shown in FIG. 8.

In other embodiments, the catheter can have a shortened length and a proximal end that is designed for placement in a guide catheter or the like during use with its distal end extending past the distal end of the guide catheter. As noted above, when used for aspiration, we refer to these catheters herein as distal access or guide supported aspiration catheters. A control structure, such as a wire, extends from the catheter and, in use, generally exits the guide catheter external to the patient. While a suitable control structure can take various forms, a wire provides a desirable small cross section and can provide appropriate control function. Regardless of the cross sectional shape of the control structure/wire, it should be fixedly attached to the catheter to avoid detachment during use and to provide appropriate control of the catheter delivery, without significantly interfering with the open lumen of the catheter.

To form a secure attachment, the control structure, such as a metal wire, can be welded or soldered to the LCH, which can be designed to facilitate the attachment. Other suitable attachments with adhesives, polymer embedding, or the like can also be used or as an alternative. FIG. 9 shows schematically one such attachment. FIG. 12 depicts a further fragmentary side view of the embodiments showing an angled opening 1102 into the catheter lumen to provide a larger surface area opening. To form a secure bond with the LCH 1100, the end of the control structure 1104 can be flattened or otherwise shaped, for example, to increase the surface area of the connection with the LCH 1100 as well as to control the profile at the connection. The overlapping length of the control structure/wire 1104 and the LCH 1100 can be from about 0.0025 in. to about 1 in. and in further embodiments from about 0.05 in to about 0.5 in. After securing the control wire 1104 to the LCH 1100, the circumference of the catheter at the attachment region 1106 may or may not be approximately circular. When the polymer jacket and optionally additional thicknesses of polymer to build up an interface section are applied, they may be molded around or slit to partially or more fully expose or reduce coverage of attachment location of the control wire with the LCH. A non-circular cross-sectional profile can provide advantages for interfacing with the inner surface of a guide catheter as described in U.S. Pat. No. 10,478,535 to Ogle (hereinafter the ‘525’ patent), entitled “Suction Catheter Systems for Applying Effective Aspiration in Remote Vessels, Especially Cerebral Arteries,” incorporated herein by reference.

The distal end of a distal access (guide assisted) aspiration catheter has an interface section with a greater external diameter to interface with the inner surface of a guide catheter to form an effective seal to facilitate fluid transfer. The diameter is selected to be suitable to form the interface and can be fixed to interface with a particular guide catheter design or adjustable to fit with a range of guide catheter designs, as described further below. Forming the interface section can be coordinated with the placement of a polymer jacket and any coatings.

It is generally desirable to angle the proximal end of the short length catheter and/or to flare the catheter to facilitate delivery of devices into the lumen to place tools in the lumen or vessel distal to the catheter. A flare of the opening should account for the limits provided by the diameter of the interface section. See FIGS. 13 and 14 which depicts an embodiment with flaring at the distal 1202 and proximal ends 1204. The LCH 1200 can be cut to both form an angle and/or distended to form the flare 1206, i.e., widening diameter, and the LCH 1200 can also be cut to form a connection point 1208 with the control wire 1210, such as at or near the top of an angled opening. A specific embodiment is described further below.

The attachment of the control structure/wire to the LCH is generally performed prior to connection with a polymer jacket and/or coatings. The placement of the polymer jacket and coatings can follow as described above with the materials described above. If the catheter is being used as an access catheter, no further structural features may be needed. For the formation of a guide supported aspiration catheter or other catheter providing for fluid exchange, an interface section on the catheter forms an effective seal against the interior surface of the guide catheter while allowing relative movement of the catheter within the guide catheter.

As noted above for the '535 patent, an interface section with an oval or other noncircular cross sectional shape can facilitate a reasonable seal while allowing sliding of the catheter. In some embodiments, the interface section can be formed by placement of additional layers of the polymer jacket around the particular section of the catheter. The difference between a major outer diameter and a minor outer diameter indicates the degree of asymmetry when not constrained by the guide catheter and may have a value from about 30 microns to about 160 microns. The lateral extent of the interface section along the length of the catheter can be from about 5 millimeters to about 25 centimeters. While the structures in the '535 patent provide desirable function, they are designed to work with a specific guide catheter size. Extendable interface sections are described in published U.S. patent application 2023/0248377 to Wainwright et al., entitled “Suction Catheter Systems With Designs Allowing Improved Aspiration and Evaluation of Aspiration Condition,” incorporated herein by reference and attached hereto as an Appendix. The interface section, referred to as a connection section, can have designs based on radially extendable structures using mechanical frame that can be actuatable or self-extending with constraint release. Actuation of extension of an interface section can be achieved with an actuation cable that can be the same or distinct from the control structure. In some embodiments, actuation cable can comprise an electrical wire or, in other embodiments, a corewire and an overtube. A radially extendable interface section can provide a tight seal upon actuation and for relative ease in movement of the guide supported aspiration catheter when not extended. These various structures for a connecting section of the distal access aspiration catheter can be formed over the LCH and connected control structure. A person of ordinary skill in the art will recognize that additional ranges of connection section dimensions within the explicit ranges above are contemplated and are within the present disclosure.

As noted above, one suitable application for the catheter design is for aspiration catheters. Aspiration thrombectomy has been found to be a useful tool for removal of thrombus from a patient's blood vessels. There is particular interest in performing aspiration thrombectomy for the treatment of acute stroke. The rapid removal of thrombus to remove clots causing an ischemic stroke event can have a dramatic influence on the achievement of good outcomes. Several aspiration catheters are currently on the market specifically for the treatment of stroke. The present designs are based to some degree on Applicant's existing aspiration catheter designs.

For the full length catheter or the guide supported aspiration catheter, the aspiration lumen is connected at a hub to fittings that generally comprise manifolds with appropriate valves, optional sensors, and other instrumentalities, such as fluid sources, a negative pressure source, such as a pump, or the like. Generally, the fittings comprise a hemostatic valve to isolate the sterile environment open to the patient's vasculature and to avoid undesirable blood loss. As appropriate other instruments can be introduced through the hemostatic valve for delivery into the patient.

Applicant has provided various improvements to the fittings to assist with aspiration thrombectomy procedures especially for ischemic stroke treatment. In particular, pressure sensors and/or flow sensors can provide valuable information on the clot extraction process and status of the clot with respect to ingestion into the catheter and ingestion of the clot into the distal fittings and tubing elements that may comprise a filter or the like to ultimately catch the clot. More automated reliance on pressure and/or flow sensor readings are described in the '518 application cited above, and in some embodiments, an automated valve, such as a solenoid valve, can be controlled automatically based on pressure and/or flow measurements in real time. The catheters described herein are designed to be effectively used with these fittings and related instrumentalities. The '518 Application also describes the associated procedures in detail.

As noted above, an infusion/balloon catheter can incorporate an LCH based catheter for the delivery of pressurized fluid. Applicant has developed desirable designs of a balloon-catheter based device for delivery of pressurized fluid into a vessel proximal to the balloon. These devices are designed for the delivery of moderate amounts of fluid at pressures to flush a vessel. While these devices in principle can be used in a range of contexts, they were specifically intended for use with an aspiration catheter to set up hydraulic forces in the vessel to facilitate clot removal. These devices are described generally in the '445 patent, cited above. The LCH based catheters described herein can be adapted for various catheter components described in the '445 patent.

An improved version of a single lumen embodiment of a hydraulic displacement catheter is presented in the '134 application cited above. The catheters described herein can be used for the main catheter body of this single lumen infusion catheter. Specifically, the LCH catheter with an embedded polymer seal, as described herein can be effectively used for the catheter body with other components appropriately attached. A specific embodiment is described further below. In these embodiments, it may be desirable to use a soft polymer liner. The liner can facilitate maintaining the flow within the catheter under pressure without delaminating.

### Specific Prototypes

Specific prototypes can be described for a full length aspiration catheter and a guide supported (distal access) aspiration catheter. The full length aspiration catheter prototype can also be adapted for use as a component of a balloon/infusion catheter with modification described in detail below. Flow testing has been performed for the guide supported (distal access) aspiration catheter prototype. Some results are described for the flow testing in the following.

A full-length aspiration catheter prototype has a LCH core is made with a laser cut hypotube divided into five distinct sections, section A, section, B, section C, section D, and section E, which is similar to the catheter in FIG. 10 except for the distinctions in the segmentation. The hypotube has an inner diameter of about 0.077 inches and an outer diameter of about 0.081 inches. Sections A, C, and E have a consistent laser cut pattern, and Sections B and D have a transitional cut pattern to transition between two adjacent sections with a consistent laser cut pattern. Transitional sections may have a generally linear progression across the section from a first pattern to a second pattern. They could also be stepwise or have non-repeating sections, as desired. Section A is at the distal end of the catheter, and section E is at the proximal end of the catheter.

The overall transition of the laser cuts are intended to involve greater flexibility of the catheter at the distal end relative to the flexibility at the proximal end. In the prototype, section A has an axial length of about 2.4 inches. The cut pattern for section A has a consistent pitch of 0.0035 inches, cuts extending 220 degrees, uncut portions extending 20 degrees, and 1.5 cuts per revolution (CPR). Section A includes an uncut distal tip extending 0.005 in. Section B has an axial length of about 5.5 inches. The cut pattern for section B has a pitch transitioning from 0.0035 inches at a distal end to 0.0065 inches at a proximal end, cuts transitioning from 220 degrees at a distal end to 154 degrees at a proximal end, uncut portions transitioning from 20 degrees at a distal end to 40 degrees at a proximal end, and 1.5 CPR at a distal end to 1.85 CPR at a proximal end. Section C has an axial length of 6.0 inches. The cut pattern for section C has a pitch of 0.0065 inches, cuts extending 154 degrees, uncut portions extending 40 degrees, and 1.85 cuts per revolution (CPR). Section B smoothly transitions from the cut pattern of Section A to the cut pattern for Section C. Section D has an axial length of about 12.0 inches. The cut pattern for section D has a pitch transitioning from 0.0065 inches at a distal end to 0.010 inches at a proximal end. The cut pattern for section D has consistent cuts extending 154 degrees, consistent uncut portions extending 40 degrees and consistent 1.85 CPR across the entire section. Section E has an axial length of about 28 inches. The cut pattern for section E has a consistent pitch of 0.010 inches, cuts extending 154 degrees, uncut portions extending 40 degrees, and 1.85 cuts per revolution (CPR). The pitch in section D smoothly transitions between the values in adjacent sections. Section E has an uncut proximal tip with a length of about 0.020 inches.

This specific embodiment of an LCH-based catheter body provides a feel for a suitable relationship of the various parameters for a spiral cut catheter body. Of course, the full range of cut parameters can be exploited to achieve desirable catheter properties. A sealing polymer can be applied and reflowed for embedding as described above. The balloon/infusion catheter has a catheter body with a port near the distal end of the catheter. This is described further below, and modifications to the catheter to accommodate the port is described below.

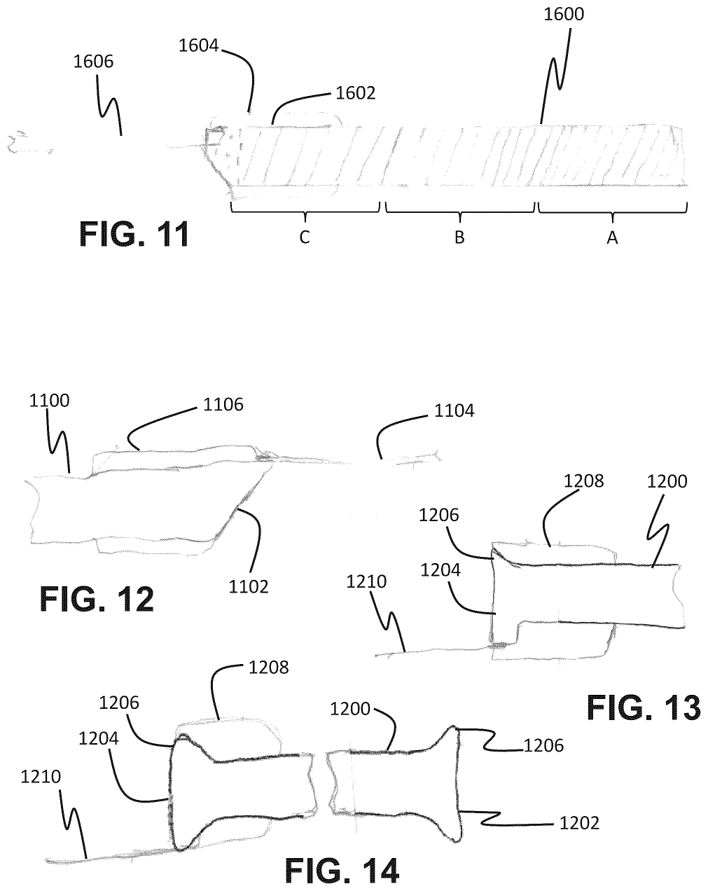

A distal access (guide supported aspiration) catheter prototype has a laser cut hypotube 1600 divided into three distinct sections, section A, section, B, and section C. See e.g. FIG. 11. For this protype embodiment, the hypotube 1600 has an inner diameter of about 0.077 inches and an outer diameter of about 0.081 inches. Sections A and C have a consistent laser cut pattern, and section B had a transitional cut pattern to transition between the two consistent sections. Transitional section B had a generally linear transition across the section. Section A includes a distal end of the catheter, and section C includes a proximal end of the catheter. Section A has an axial length of about 2.4 inches. The cut pattern for section A has a consistent pitch of 0.0035 inches, cuts extending 220 degrees, uncut portions extending 20 degrees, and 1.5 cuts per revolution (CPR). Section A has an uncut distal tip with an axial length of about 0.005 inches. Section B has an axial length of about 5.5 inches. The cut pattern for section B has a pitch transitioning from 0.0035 inches at a distal end to 0.0065 inches at a proximal end, cuts transitioning from 220 degrees at a distal end to 154 degrees at a proximal end, uncut portions transitioning from 20 degrees at a distal end to 40 degrees at a proximal end, and 1.5 CPR at a distal end to 1.85 CPR at a proximal end. Section C has an axial length of 1.8 inches. The cut pattern for section C has a consistent pitch of 0.0065 inches, cuts extending 154 degrees, uncut portions extending 40 degrees, and 1.85 cuts per revolution (CPR). The laser cuts in each section have a kerf of 0.001 inches. Again, these prototype values give an example for suitable cut parameters and transitions to improve flexibility. Of course, other values of specific cut parameters can be used using the guidelines above to achieve desired amounts of flexibility.

Section C abuts, at the proximal end 1602, a section for attachment 1604 of the control structure 1606 such that an overall axial length of the Q catheter is about 9.8 inches. In some embodiments, the section for attachment 1604 of the control structure 1606 may have a cut corresponding to a shape of a distal portion of the control wire 1606. In the prototype, the connecting section has a generally rectangular cut with an open proximal end to receive a distal portion of the core wire, see FIG. 9 and a fragmentary tip view in FIG. 17A. In other embodiments, hypotube can be laser cut for wire attachment with a disc shaped cut 1702 (flattened view in FIG. 17B), a puzzle piece shaped cut 1704 (flattened view in FIG. 17C), or the like. Section 1604 may have a further polymer The core wire is flattened (or welded or otherwise secured to a flattened element) and affixed to the connecting section by welds. The connection section has small, regularly spaced, laser cut holes for improved flexibility. For this portion of the connection section, spiral cuts can be impractical due to the modifications for the control wire attachment.

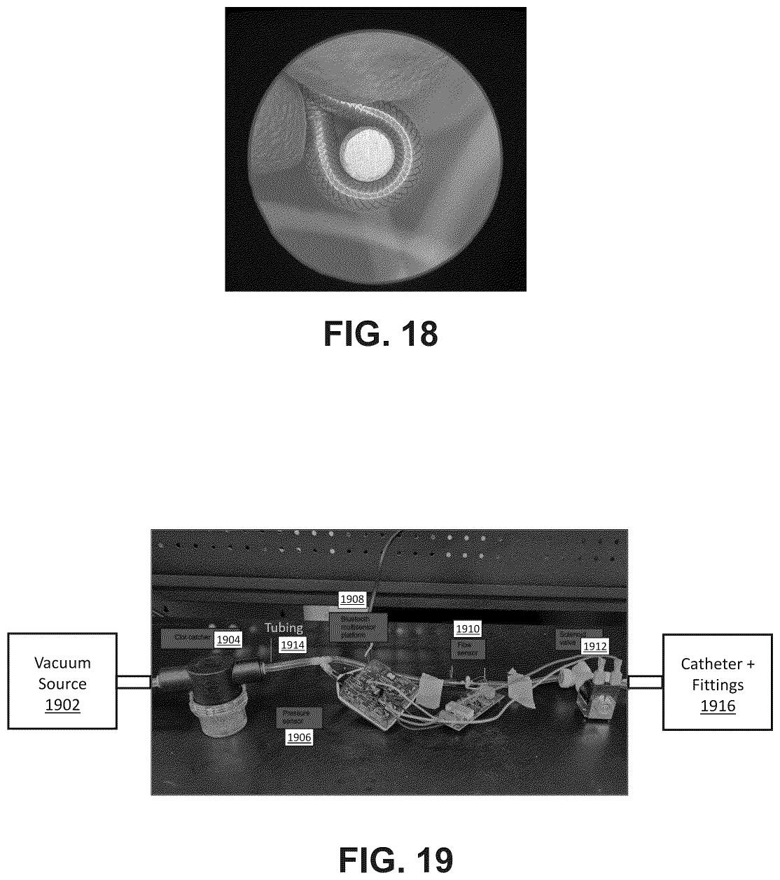

The distal end of the hypotube extending roughly 0.5 in was stretched to increase flexibility. To form the prototype catheter, soft polyurethane polymer was used for a liner and for a polymer jacket. With the liner and jacket, the inner diameter was about 0.072 in and the outer diameter was about 0.086 in, which corresponded to about 0.005 in reduction in inner diameter and 0.005 in increase in outer diameter relative to the LCH. The wall thickness was about 0.007 in. A metal marker band was bound adjacent the distal end with a small overhang of polymer extending past the marker band. The catheter was bendable around a 3 mm mandrel as shown in FIG. 18.

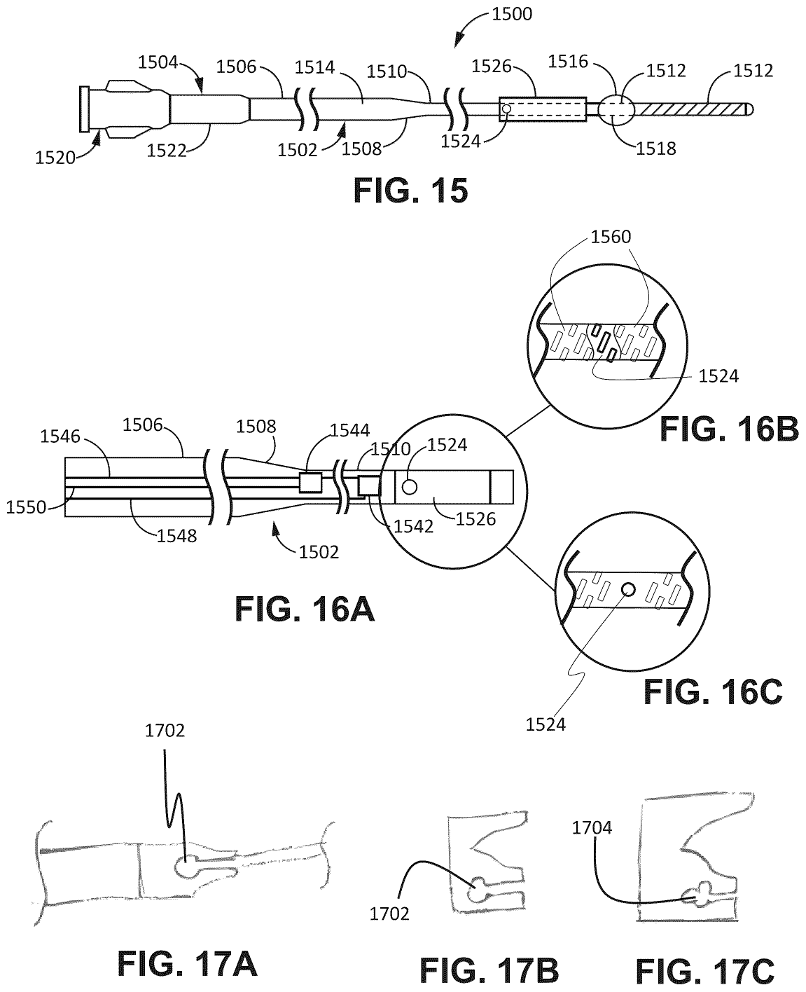

Referring to FIG. 15, a single lumen occlusion balloon/infusion catheter 1500 comprises tubular shaft 1502 forming lumen 1514, balloon 1516, corewire 1518 floating within lumen 1514 and extending from a distal end of tubular shaft 1502, and a proximal fitting 1504. Referring to the embodiment of FIGS. 15 and 16, tubular shaft 1502 comprises a proximal section 1506, distal section 1510 with a smaller diameter than proximal section 1506 and a transition section 1508 connecting proximal section 1506 and distal section 1510. Transition section 1508 can have an abrupt diameter change or a gradual transition of diameter, such as a linear transition. Proximal section 1506 connects with proximal fitting 1504. In alternative embodiments, tubular shaft 1502 can have a constant outer diameter, or more than two sections with differing constant diameters and corresponding connecting sections. Tubular shaft 1502 can be formed from laser cut hypotube with an embedded polymer seal formed from a reflowed polymer jacket, reflowed polymer liner, fluid applied and cured polymer, or combinations thereof.

Balloon 1516 can be a compliant balloon and has an interior in fluid communication with lumen 1514 such that adjustment of fluid pressure within lumen 1514 can expand or deflate balloon 1516. Corewire 1518 internal to the catheter can provide characteristics similar to a guidewire for the catheter, and a distal coil tip 1512 can be optional as long as the catheter tip is configured to avoid injury to the vessel wall. Corewire 1518 can move within a small range of motion near its proximal end (not shown) to provide for deployment of balloon 1516, which draws the distal tip 1512 toward the distal end of tubular shaft 1502 at the proximal side of balloon 1516.

Tubular shaft 1502 comprises a lumen 1514 extending from proximal fitting 1504 to balloon 1516. Lumen 1514 opens at the distal end of tubular shaft 1502 into the interior of balloon 1516, such that fluid communication is established between lumen 1514 and the interior of balloon 1516. One or more infusion port(s) 1524 can provide for infusion of liquid from lumen 1514 delivered from a liquid source attached to proximal fitting 1520 into the patient's vessel. Flow of liquid out from lumen 1514 tends to lower the pressure within the lumen. Liquid pressure within lumen 1514 also provides for inflation of balloon 1516. Also, an embodiment of the infusion structure is shown in which an elastic cover 1526, configured as a polymer valve, covers infusion ports 1524. Elastic cover 1526 can provide an added measure of control over the infusion process in which a certain amount of pressure may be applied to expand clastic cover 1526 to provide for infusion. Elastic cover 1526 can be sealed along one edge to tubular shaft 1512 and open at an opposite edge to provide for the infusion. Elastic cover 1526 can be made from the same or similar material as balloon, 1516, although as long as elastic cover 1526 provides desired elastic properties, it can be made from a distinct material.

Elastic cover 1526 functioning as a polymer valve can be a tubular piece of elastomer sealed on one edge that opens as a check valve when the pressure is sufficient. Of course, non-tubular polymer sheets can be sealed along multiple edges with an open edge to provide a similar effect. The valve pressure response can be designed through the polymer and thickness, the length of the polymer tube relative to the sealed edge, with a longer tube eliciting a greater pressure to open the valve, and/or the any tension in the polymer, such as stretching the polymer over the catheter tube, if the relaxed polymer tube has a smaller diameter than the catheter tube. These design parameters can be adjusted to provide for e polymer valve to open at an appropriate pressure to expand the balloon. The balloon can be preconditioned by inflating and deflating the balloon during manufacturing. The preconditioned balloon can more easily inflate during a procedure. The balloon and polymer valve can be cut form a common extruded elastomer tube with the other design parameters used to adjust their responses during the procedure, although the properties can be adjusted to be different for the two components. In some embodiments, the elastic cover/polymer valve 1526 can have a length from about 0.1 inches to about 2 inches, and in further embodiments from about 0.15 inches to about 1.5 inches. The balloon can have an unexpanded length from about 0.1 inches about 1 inch. A person of ordinary skill in the art will recognize that additional ranges of polymer element lengths along the catheter within the explicit ranges above are contemplated and are within the present disclosure.

The hydrodynamics should be balanced so that appropriate pressures to maintain the inflated balloon while correspondingly to provide for a desired infusion flow rate. Infusion port(s) 1524 and elastic cover 1526, if present, can be designed accordingly, and infusion port(s) 1524 are also in appropriate proximity to balloon 1516 to facilitate placement distal to the clot within tortuous vessels. In particular, the size and number of infusion port(s) 1524 can be selected to provide appropriate infusion at pressures inflating balloon 1516. In some embodiments, the farthest edge of an infusion port is no more than 5 centimeters from the closest edge of the balloon, and in further embodiments no more than 2.5 centimeters, and this spacing is general for all of the catheter embodiments in this section. A person of ordinary skill in the art will recognize that additional values of infusion port spacing within the explicit ranges above are contemplated and are within the present disclosure.

To facilitate monitoring of the pressures and flows through the system, the balloon/infusion catheter can be instrumented with sensors. One embodiment would be using an adjustable pressure infusion syringe pump or pressure bag or other infusion pump to provide a controllable volume and/or flow rate for the infusion. Various pressure infusion syringe devices are commercially available, such as Masterflex® Infusion Pump, Medfusion® Wireless Syringe Infusion Pump, a Merit Medical Pressure Infusor Bag™, ASP Medical Pressure Infusor Bags, and the like. Other pumps can also be adapted for these purposes as desired. This would allow the infusion pressure to be set and maintained. Referring to FIG. 16A, tubular shaft 1502 can be provided with one or more pressure sensors. Pressure sensor 1542 is positioned to measure the pressure between the balloon and the original position of a clot, and pressure sensor 1544 is positioned to measure the pressure between the original position of the clot and an aspiration catheter. Wiring for the sensors can be embedded within a polymer wall or otherwise tracked along the length of the tubular shaft to an exterior location. For example, as shown in FIG. 16A, ground wire 1546 connects to pressure sensor 1542, 1544, and wires 1548, 1550 connect to pressure sensors 1542, 1544 respectively. Various suitable pressure sensors can be used. Integrated circuit pressure sensors can be used such as the Infineon KP236 pressure sensor. A piezo resistive pressure die P330 W is available from Nova®Sensor with a thickness of 120 microns. The use of these pressure sensors in a catheter is described in published U.S. patent application 2018/0010974A to Bueche et al., entitled “Pressure Sensor System,” incorporated herein by reference. Optical pressure sensors can also be used in all the various portions of the device similar to the Boston Scientific Comet II Guidewire. The pressure readings could also be transmitted wirelessly like the Abbott Pressurewire X guidewire. Balloon 1516 can be a tubular/cylindrical section of polymer that is connected at both ends to isolate the interior, although other topologically equivalent structures can be used to secure the balloon and isolate the interior. One edge of the balloon can be secured around tubular shaft 1512 and the other edge can be secured around the corewire. The edges of the balloon can be secured using heat bonding, adhesive bonding, fastening with a band, combinations thereof, or the like. The fastening of the balloon to tubular shaft 1512 at one end and floating corewire 1518 at the other end advantageously permits balloon 1516 to neck down when the catheter is advanced proximally or distally within the vessel, allowing the catheter system to more easily maneuver through the vasculature. Sliding of corewire 1518 further permits rapid inflation or deflation of balloon 116 through the combined effect of fluid pressure and mechanical movement of the secured edges of the balloon relative to each other.

To accommodate port 1524, several embodiments are discussed. In a first approach shown in FIG. 16B, polymer 1560 is excluded from the region of the fluid port 1524. Exclusion of the polymer 1560 can be achieved during formation, such as using a jacket with a preformed hole, applying the polymer by brushing or the like with the hole masked off or otherwise excluded, or any similar approach. In other embodiments, polymer can be ablated chemically or with an appropriate form of radiation to remove the polymer from the area of the port. If polymer is excluded, fluid can flow from the interior of the catheter through the laser cuts such that the port 1524 is identifiable by the exclusion of polymer. Similarly, the port 1524 can be formed as a fully open region through the LCH catheter wall. See FIG. 16C. For such embodiments, the immediate section of LCH around the port 1524 can avoid a spiral cut. So various shaped cuts can be used in the vicinity of the port for flexibility, such as a pattern of holes. The polymer still should be excluded from covering the port using a technique from about in this paragraph.

### Bench Testing of LCH-Based Distal Access Aspiration Catheter

An experimental system configured to allow for real time monitoring of a simulated aspiration thrombectomy used in the trials described below is illustrated in FIG. 19 and is described in the '518 application cited above. The system generally included a vacuum source 1902, a clot catcher (filter) 1904, a pressure sensor 1906, a controller 1908, a flow sensor 1910, a valve 1912, extension tubing 1914 and an aspiration catheter+fittings 1916.

The vacuum source 1902 was a vacuum pump configured to operate at a pressure of about −30 inHg. The vacuum source 1902 was fluidly connected to the aspiration catheter 1916 by extension tubing 1914 and valve 1912. The pressure sensor 1906 was fluidly connected between the vacuum source 1902 and aspiration catheter 1916 and was configured to measure real time pressure of the fluid passing through the extension tubing 1914. The measurements were communicated to controller 1908.

The clot catcher 1904 was fluidly connected between the vacuum source 1902 and pressure sensor 1906 and sized appropriately to capture simulated clots while letting fluid flow essentially unimpeded. A soft Thrombotech™ synthetic clot obtained from Biomedix was used to simulate a red blood cell clot. The clot was sized to have a diameter of about 2.0 mm. and was positioned in silicone tubing to simulate a blood vessel. The silicone tubing had an ID of 1.5 mm or 1.6 mm.

The flow sensor 1910 included a printed circuit board electrically connected to unheated and heated temperature sensors. The printed circuit board was configured to measure the real time flow rate using the constant temperature anemometer principle (i.e., the amount of heat removed from the heated temperature sensor by a flowing fluid is related to that fluid's velocity with the unheated temperature sensor being used to compensate for variations in the air temperature). The measurements were communicated to controller 1908 by an electrical wire.

Valve 1912 was a solenoid valve electronically connected to controller 1908 and was configured to open and close based on signals sent from controller 1908. The controller 1908 was configured to receive input regarding measurements from pressure sensor 1906 and flow sensor 1910 and communicate output to a display and to the valve 1912 for the purpose of opening and closing the valve. Tubing running through solenoid valve 1912 to pump 1902 connected to catheter (+fittings) 1916.

For the trials, a soft Thrombotech™ synthetic clot, cut to a length of about 10 mm, was positioned in the silicone tubing using a syringe. The aspiration catheters that were tested included a Q™ 6 catheter and a catheter according to the present disclosure (identified as “HerQles™”) with the catheter's inner, and in some cases outer diameters, either uncoated or coated with a polymer. The Q6 catheter and the HerQles™ catheter were similar other than the replacement of the LCH based catheter body of the HerQles catheter replacing the polymer catheter body of the Q6 catheter and had an outer diameter of about 6 Fr. The HerQles™ catheter was the prototype guide assisted aspiration catheter described above. A full length aspiration catheter with polymer tubing was also tested.

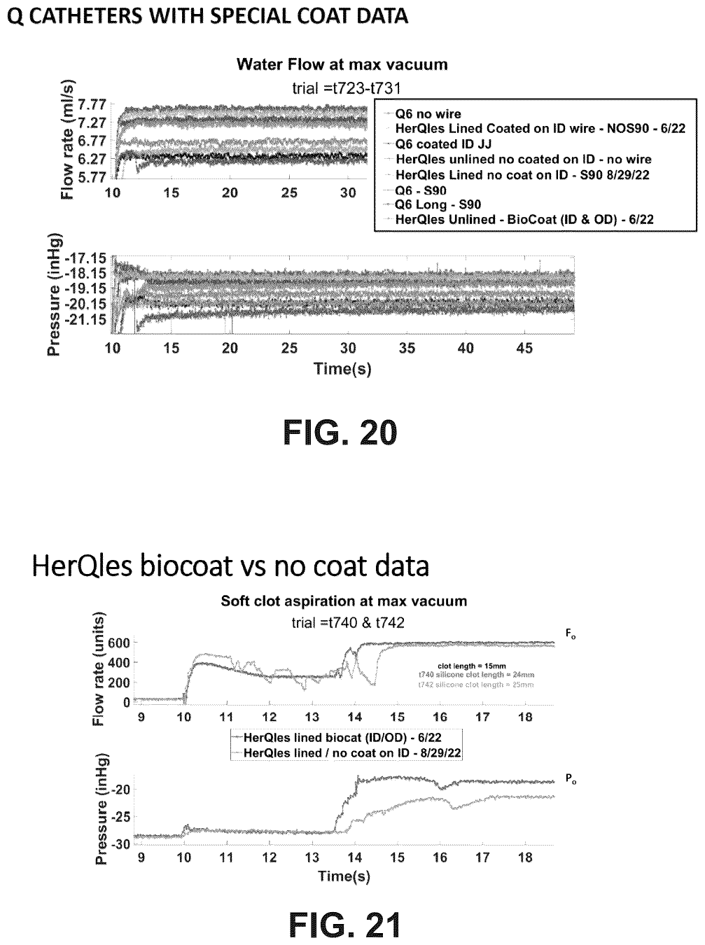

The basic flow properties of eight catheters were tested using the set up with the pump and the catheter tip immersed in a water reservoir. The pressure and flow rate measurements are shown in FIG. P18 for four version of the HerQles™ catheter (lined with a coating on the inner diameter (ID), unlined and coating on outer diameter (OD), lined and coating only on OD, and unlined coating on ID and OD), three versions of the Q6 guide supported aspiration catheter (uncoated, coated on the ID, and S90), and one 6F long aspiration catheter with roughly the Q6 ID and OD values over the full length. As shown in FIG. 20, pressure Pₒ ranged from about −18 inHg to about −20 inHg. Flow rate Fₒ ranged from about 6 milliliter per second (ml/s) to about 7.5 ml/s. A higher flow rate correlates with a lower pressure since the flow results in a reduced pressure and vice versa. It is desirable to have a larger free flow rate since this suggests a stronger aspiration at the catheter tip.

Before beginning the trial with the HerQles™ catheter with a liner and ID coating and with a liner and a ID coating, the solenoid valve was closed by the controller. In order to obtain baseline pressure and flow rate values, the vacuum pump was turned on for approximately 1 minute during which time the pressure within the extension tubing was continuously measured by the pressure sensor to obtain the maximum negative pressure P_{c} available for the pump (the flow rate Fe being equal to 0). The distal tip of the catheter was then immersed in water and the solenoid valve was electronically opened to fill the system with water. The pressure and flow rate within the extension tubing was then measured by the pressure sensor and flow sensor to obtain an open control pressure Pₒ and steady state flow rate Fₒ of water through the unconstrained catheter.

The solenoid valve was then electronically closed for approximately 10 seconds to remove any air within the system. The HerQles™ catheter was then passed through a hemostatic valve and its distal end directed to the clot face within the silicone tubing. Once the catheter was in place, the solenoid valve was opened and the pressure and flow rate within the extension tubing was continuously measured by the pressure sensor and flow sensor. The measured pressures and flow rates are plotted as a function of time in FIG. 21. During this trial, the maximum negative pressure P_{c} was about −29 inHg. After 10 seconds, the valve was opened, and the negative pressure increased to about −27 inHg and remained steady while the flow rate varied between about 50-60% of Fₒ for approximately 3.5 seconds for the catheter having a polymer coating and 4 seconds for the uncoated catheter suggesting the clot was corked. The negative pressure and flow rate then began to increase to Pₒ and Fₒ and remained steady suggesting the clot was corked at the catheter tip. At about 13 seconds for the catheter with an inner coating and at about 14 seconds for the uncoated catheter the sensor readings suggested the clot was travelling through and clearing from the system. Both HerQles™ catheters quickly ingested and cleared the clot from the system with the polymer coated catheter ingesting and clearing the clot slightly faster than the uncoated catheter.

### Further Inventive Concepts

A1. A distal access aspiration catheter comprising:

a laser cut hypotube having a distal end and a proximal end, and forming a tubular element with an aspiration lumen and a connection section for interfacing with a guide catheter to complete an aspiration lumen extending through the proximal portion of the guide catheter;

an embedded polymer tube flowed into the cuts of the laser cut hypotube; and

a control wire comprising a distal end welded or soldered to a matching configuration in the structure of the hypotube in the connection section at or near the proximal end of the laser cut hypotube.

A2. The distal access aspiration catheter of inventive concept A1 wherein the proximal end of the laser cut hypotube comprises an angled opening into the aspiration lumen.
A3. The distal access aspiration catheter of inventive concept A1 wherein an overlapping portion of the control wire and the laser cut hypotube is about 0.0025 inches to about 1 inches in length.
A4. The distal access aspiration catheter of inventive concept A1 wherein the distal end of the control wire is flattened and the proximal end of the hypotube comprise a corresponding mated pattern configured to interface with the flattened distal end to prevent separation of the control wire and hypotube.
A5. The distal access aspiration catheter of inventive concept A1 wherein the laser cut hypotube comprises a plurality of sections, each of the plurality of sections having a distinct laser cut pattern.
A6. The distal access aspiration catheter of inventive concept A5 wherein the plurality of sections have greater flexibility in a distal direction.
A7. The distal access aspiration catheter of inventive concept A5 wherein the plurality of sections comprises three section wherein a proximal section and a distal section have constant laser cuts along their length and a middle section transitions the laser cuts between the proximal section and the distal section and wherein the distal section has greater flexibility than the proximal section.
A8. The distal access aspiration catheter of inventive concept A1 wherein the laser cut hypotube comprises a flared distal end, a flared proximal end or both a flared distal end and a flared proximal end.
A9. The distal access aspiration catheter of inventive concept A1 wherein a section of the laser cut hypotube comprises a spiral laser cut with a consistent pitch.
A10. The distal access aspiration catheter of inventive concept A1 wherein a transitional section of the laser cut hypotube comprises a generally linear transition of a spiral cut parameter across the transitional section.
A11. The distal access aspiration catheter of inventive concept A1 having a length from about 1.18 inches (3 cm) to about 23.6 inches (60 cm) and an average outer diameter away from the connection section from about 3 Fr (1 mm) to about 6.5 Fr (2.17 mm).
A12. The distal access aspiration catheter of inventive concept A1 wherein the distal end of the laser cut hypotube is flared.
A13. The distal access aspiration catheter of inventive concept A12 wherein the flared end has a length of no more than about 0.197 inches (5 mm).
A14. The distal access aspiration catheter of inventive concept A1 wherein the distal end of the laser cut hypotube has an extending section configure to expand into a funnel.
A15. The distal access aspiration catheter of inventive concept A1 further comprising a radiopaque marker band that is laser cut or comprises radiopaque wire, and wherein the embedded polymer further embeds the radiopaque marker band.
A16. The distal access aspiration catheter of inventive concept A1 wherein the polymer has a Shore Durometer value from about 35 A to about 76 A,
A17. The distal access aspiration catheter of inventive concept A1 wherein the laser cut hypotube with embedded polymer has an average wall thickness from about 0.0020 inches (0.0508 mm) to about 0.010 inches (0.254 mm).
B1. A infusion balloon catheter comprising:

a laser cut hypotube with a port through the wall near the distal end of the hypotube;

an embedded polymer flowed into the cuts of the laser cut hypotube;

a balloon in fluid communication with the interior of the laser cut hypotube; and

a distal tip distal to the balloon.

B2. The infusion balloon catheter of inventive concept B1 wherein the port is proximal to the balloon.
B3. The infusion balloon catheter of inventive concept B1 wherein a polymer valve covers the port.
B4. The infusion balloon catheter of inventive concept B1 wherein a distal edge of the port is no more than 5 centimeters from a proximal edge of the balloon.
B5. The infusion balloon catheter of inventive concept B1 wherein the laser cut hypotube comprises a plurality of sections, each of the plurality of sections having a distinct laser cut pattern.
B6, The infusion balloon catheter of inventive concept B5 wherein the plurality of sections comprise sections with different outer diameters and a transition section connecting two sections with different outer diameters.
B7. The infusion balloon catheter of inventive concept B5 wherein a more distal section has a greater flexibility than a more proximal section.
B8. The infusion balloon catheter of inventive concept B5 wherein at least one section has a spiral laser cut.
B9. The infusion balloon catheter of inventive concept B1 further comprising a corewire within the lumen of the laser cut hypotube.
B10. The infusion balloon catheter of inventive concept B8 wherein the corewire has a distal coil secured to a solid wire section.
B11. The infusion balloon catheter of inventive concept B9 wherein the balloon is secured at a proximal end to the laser cut hypotube and at a distal end to the corewire, and wherein a proximal end of the corewire has a range of motion to provide for relative movement of the corewire relative to the hypotube to provide for expansion and deflation of the balloon.
B12. The infusion balloon catheter of inventive concept B1 wherein the proximal end of the laser cut hypotube is secured inside of a hub.
B13. The infusion balloon catheter of inventive concept B12 wherein the laser cut hypotube is laser cut within the hub.
B14. The infusion balloon catheter of inventive concept B1 wherein the port is formed by a gap in the embedded polymer to allow fluid flow through the laser cuts
B15. The infusion balloon catheter of inventive concept B1 wherein the port is formed through a specific hole formed though the hypotube and embedded polymer.
B16. The infusion balloon catheter of inventive concept B1 wherein the polymer has a Shore Durometer value from about 35 A to about 76 A,
B17. The infusion balloon catheter of inventive concept B1 wherein the laser cut hypotube with embedded polymer has an average wall thickness from about 0.0020 inches (0.0508 mm) to about 0.010 inches (0.254 mm).

The embodiments above are intended to be illustrative and not limiting. Additional embodiments are within the claims. In addition, although the present invention has been described with reference to particular embodiments, those skilled in the art will recognize that changes can be made in form and detail without departing from the spirit and scope of the invention. Any incorporation by reference of documents above is limited such that no subject matter is incorporated that is contrary to the explicit disclosure herein. To the extent that specific structures, compositions and/or processes are described herein with components, elements, ingredients or other partitions, it is to be understand that the disclosure herein covers the specific embodiments, embodiments comprising the specific components, elements, ingredients, other partitions or combinations thereof as well as embodiments consisting essentially of such specific components, ingredients or other partitions or combinations thereof that can include additional features that do not change the fundamental nature of the subject matter, as suggested in the discussion, unless otherwise specifically indicated. The use of the term “about” herein refers to expected uncertainties in the associated values as would be understood in the particular context by a person of ordinary skill in the art.

What is claimed is:

1. A medical catheter for transfer of fluids comprising:
a laser-cut hypotube having a proximal end and a distal end; and
an embedded polymer flowed into the cuts of the laser-cut hypotube, wherein the polymer has a Shore Durometer value from about 35 A to about 76 A.

2. The medical catheter of claim 1 wherein the laser cut hypotube is configured to extend along at least about the distal 30% of the catheter length.

3. The medical catheter of claim 1 wherein the laser cut hypotube comprises a plurality of sections, each of the plurality of sections having a distinct laser cut pattern.

4. The medical catheter of claim 3 wherein at least two sections comprise a spiral laser cut and each of the distinct laser cut patterns differ from one another by differences of one or more of a pitch, a degree of an uncut portion, and a cuts per revolution.

5. The medical catheter of claim 3 wherein the distinct laser cut pattern is associated with a flexibility of the corresponding section.

6. The medical catheter of claim 1 wherein a first section of the laser cut hypotube comprises a consistent pitch and a second section of the laser cut hypotube comprises a pitch transitioning from a first value at a distal end to a second value at a proximal end.

7. The medical catheter of claim 1 wherein a section of the laser cut hypotube transitions from a first cuts per revolution at a first end to a second cuts per revolution at a second end.

8. The medical catheter of claim 1 wherein the laser cut hypotube with embedded polymer has an average wall thickness from about 0.0020 inches (0.0508 mm) to about 0.010 inches (0.254 mm).

9. The medical catheter of claim 1 wherein the laser cut hypotube with embedded polymer has an average wall thickness from about 0.0025 inches (0.0635 mm) to about 0.008 inches (0.216 mm).

10. The medical catheter of claim 1 wherein the polymer has a Shore Durometer value from about 42 A to about 58 A.

11. The medical catheter of claim 1 wherein the hypotube comprises stainless steel.

12. The medical catheter of claim 1 comprising uncut segments at a distal end and a proximal end, the uncut segments independently having a length along the catheter from about 0.0020 in to about 0.1 and at least one section of spiral cut hypotube.

13. The medical catheter of claim 1 further comprising a low friction polymer coating over an exterior surface, an interior surface, or both.

14. The medical catheter of claim 13 wherein the low friction polymer coating comprises a hydrophilic polymer.

15. The medical catheter of claim 1 wherein the proximal end of the laser cut hypotube is inserted into and secured to a hub.

16. The medical catheter of claim 1 wherein a control structure is secured to the laser cut hypotube at or near the proximal end of the hypotube and a distal end of the control structure.

17. The medical catheter of claim 16 wherein the control structure comprises a feature that interfaces with a mated feature cut into the hypotube to facilitate securing of the control structure.

18. The medical catheter of claim 16 wherein a polymer jacket is placed over the secured distal end of the control structure to form a connection section.

19. The medical catheter of claim 1 wherein the laser cut hypotube has a port near the distal end.

20. The medical catheter of claim 19 wherein the port is formed by a gap in the embedded polymer to allow fluid flow through the laser cuts, or the port is formed through a specific hole formed though the hypotube and embedded polymer.

21. The medical catheter of claim 1 wherein a difference between a catheter wall thickness and a laser cut hypotube wall thickness ranges from about zero to about 0.0015 inches (0.0381 mm).

22. The medical catheter of claim 1 further comprising a radiopaque marker band that is laser cut or comprises radiopaque wire, and wherein the embedded polymer further embeds the radiopaque marker band.
